(19) United States
(12) Patent Application Publication
(10) Pub. No.: US 2011/0008392 A1
(43) Pub. Date: Jan. 13, 2011

(54) VACCINE TARGETS AND DELIVERY SYSTEMS FOR CRYPTOSPORIDIUM

(76) Inventors: Gregory A Buck, Richmond, VA (US); Patricio A. Manque, Chile, VA (US); Fernando Tenjo, Chesterfield, VA (US); Myna Garcia Serrano, Richmond, VA (US); Joao Marcelo Pereira Alves, Richmond, VA (US); Ping Xu, Richmond, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/739,810

(22) PCT Filed: Oct. 24, 2008
(86) PCT No.: PCT/US08/81138

§ 371 Date: Aug. 18, 2010

Related U.S. Application Data
(60) Provisional application No. 60/982,744, filed on Oct. 26, 2007.
(60) Provisional application No. 60/983,249, filed on Oct. 29, 2007.
(60) Provisional application No. 60/983,593, filed on Oct. 30, 2007.

(51) Int. Cl. A61K 39/002; A61K 38/16; A61K 31/7052; A61P 37/04; A61P 33/02

(52) U.S. Cl. 424/269.1; 514/21.2; 514/4.4; 514/44 R

(57) ABSTRACT

Compositions comprising the Cryptosporidium sporozoite antigens such as SRK (‘similar to riken’), CP15 and profilin are used in vaccines against the protozoan parasite Cryptosporidim.

### BACKGROUND OF THE INVENTION

1. Field of the Invention

The invention generally relates to vaccines against the protozoan parasite Cryptosporidium. In particular, the invention provides vaccines and vaccine delivery systems based on the Cryptosporidium antigens SRK, CP15 and profilin.

2. Background of the Invention

Cryptosporidiosis, typically caused by the ubiquitous protozoan parasite Cryptosporidium hominis or Cryptosporidium parvum, is a leading cause of acute, persistent and chronic diarrhea worldwide (1). The Environmental Protection Agency estimates that 2.1-4.3 million cases of cryptosporidiosis occur annually in the United States alone (2), and Cryptosporidium is the most common cause of diarrhea caused by recreational water. Cryptosporidiosis is a very severe problem in developing countries, where it causes an estimated 30% of the chronic diarrhea in children under the age of three. Furthermore, cryptosporidiosis has a devastating, often lasting impact on immunocompromised or malnourished individuals (5).

Cryptosporidium does not utilize an insect vector and is capable of completing its life cycle within a single mammalian (e.g., human or animal) host, resulting in cyst stages which are excreted in feces and are capable of transmission to a new host. The pathogen is spread by contaminated water and food, through exposure to infected animals and by fecal-oral contact. Many features of Cryptosporidium enhance its infectivity and potential to cause widespread outbreaks. Infectious oocysts are ubiquitous, small (4-6 microns), hardy and resistant to many chemical disinfectants (3). As demonstrated by the >400,000 persons affected in the largest recorded water-borne outbreak in the history of the United States, Cryptosporidium species have the ability to sicken a large proportion of the population after contamination of a point source of water (4). In fact, Cryptosporidium is classified as a Class B Agent of Bioterrorism.

Cryptosporidium is also an agricultural problem, infecting pigs, calves and other mammals, and having a significant economic impact in agriculture. It is also thought that much of the accidental contamination of lakes, rivers, and water supplies is due to contamination with the feces of infected farm animals.

The study of the human immune response to cryptosporidiosis is in its infancy. The genomes of Cryptosporidium parvum (11), and Cryptosporidium hominis (9) became available only in 2004. Unfortunately, there are currently no effective vaccines against this disease. Past efforts to develop vaccines have focused largely on immunodominant antigens identified through traditional approaches. Unfortunately, such vaccines are inherently flawed because, by definition, as immunodominant antigens they are often hypervariable and change rapidly as a result of parasite evolution. Such antigens constitute a “moving target” for the immune system, and are thus compromised as vaccine targets.

The development of vaccines that protect against cryptosporidiosis is clearly a desideratum from the point of view of those who treat infectious diseases in both humans and animals, and from the standpoint of national security. It would be particularly useful to develop vaccine targets using a fresh approach to antigen selection.

### SUMMARY OF THE INVENTION

The present invention provides Cryptosporidium vaccines that elicit strong humoral and cellular immune responses to the antigens contained therein, in subjects to whom they are administered. The antigens that were selected as vaccine targets are, counter-intuitively, not based on immunodominant proteins. Therefore, these antigens have not been subject to heavy selection by natural immune systems, making them ideal targets for vaccines. In addition, the antigens are all expressed in sporozoites, which are the infectious form of the parasite and therefore believed to be the best targets for a vaccine. The antigens include the SRK antigen; the CP15 antigen; and the profilin antigen.

The invention provides compositions comprising one or more recombinant amino acid sequences as set forth in SEQ ID NO: 1, SEQ ID NO: 3, and SEQ ID NO: 5, or variants thereof; or one or more nucleic acid sequences encoding said one or more recombinant amino acid sequences of said variants; and a physiological compatible carrier. The nucleic acid sequences may be present within a vector, e.g. a Salmonella based vector such as pSEC 10 ClyA.

The invention also provides a method of vaccinating a subject against Cryptosporidiosis. The method comprises the step of 1) providing to said subject a composition comprising one or more recombinant proteins with amino acid sequences set forth in SEQ ID NO: 1, SEQ ID NO: 3, and SEQ ID NO: 5, or variants thereof, and a physiologically compatible carrier; or 2) providing to said subject a composition comprising one or more nucleic acid sequences encoding said one or more recombinant proteins with amino acid sequences set forth in SEQ ID NO: 1, SEQ ID NO: 3, and SEQ ID NO: 5, or variants thereof, and a physiologically compatible carrier; or 3) sequentially providing to said subject i. a composition comprising one or more nucleic acid sequences encoding said one or more recombinant proteins with amino acid sequences set forth in SEQ ID NO: 1, SEQ ID NO: 3, and SEQ ID NO: 5, or variants thereof, and a physiologically compatible carrier; and ii. a composition comprising one or more recombinant proteins with amino acid sequences set forth in SEQ ID NO: 1, SEQ ID NO: 3, and SEQ ID NO: 5, or variants thereof, and a physiologically compatible carrier. The composition is or said compositions are provided in a quantity sufficient to protect said subject against infection by Cryptosporidium, or to lessen symptoms of Cryptosporidiosis is said subject. The nucleic acid sequences may be present within a vector, e.g. a Salmonella based vector such as pSEC 10 ClyA, which may be administered intranasally.

The invention further provides a method of decreasing the shedding of Cryptosporidium oocysts by a subject infected with Cryptosporidium. The method comprises the step of 1) providing to said subject a composition comprising one or more recombinant proteins with amino acid sequences set forth in SEQ ID NO: 1, SEQ ID NO: 3, and SEQ ID NO: 5, or variants thereof, and a physiologically compatible carrier; or 2) providing to said subject a composition comprising one or more nucleic acid sequences encoding said one or more recombinant proteins with amino acid sequences set forth in SEQ ID NO: 1, SEQ ID NO: 3, and SEQ ID NO: 5, or variants thereof, and a physiologically compatible carrier; or 3) sequentially providing to said subject i. a composition comprising one or more nucleic acid sequences encoding said one or more recombinant proteins with amino acid sequences set forth in SEQ ID NO: 1, SEQ ID NO: 3, and SEQ ID NO: 5, or variants thereof, and a physiologically compatible carrier; and ii. a composition comprising one or more recombinant proteins with amino acid sequences set forth in SEQ ID NO: 1, SEQ ID NO: 3, and SEQ ID NO: 5, or variants thereof, and a physiologically compatible carrier; wherein said composition is or said compositions are provided in a quantity sufficient to reduce the number of Cryptosporidium oocysts shed by said subject. The nucleic acid sequences may be present within a vector, e.g. a Salmonella based vector such as pSEC 10 ClyA, and may provided intranasally.

The invention further provides a composition for use as an adjuvant, comprising a profilin protein with an amino acid sequence as set forth in SEQ ID NO: 5, or a nucleic acid sequence encoding said protein with an amino acid sequence as set forth in SEQ ID NO: 5, or a variant thereof; and a physiologically compatible carrier. The invention further provides a method of increasing an immune response to an antigen in a subject in need thereof. The method comprises the step of providing to said subject i) said antigen of interest or a nucleic acid encoding said antigen of interest; and ii) a profilin protein with an amino acid sequence as set forth in SEQ ID NO: 5 or a variant thereof, or a nucleic acid sequence encoding said profilin protein with an amino acid sequence as set forth in SEQ ID NO: 5 or a variant thereof; wherein said protein with an amino acid sequence as set forth in SEQ ID NO: 5 or a variant thereof thereof or said nucleic acid sequence encoding said protein with an amino acid sequence as set forth in SEQ ID NO: 5 or or a variant thereof thereof is provided in a quantity sufficient to cause an increase in said immune response to said antigen of interest in said subject.

The invention also provides a method of vaccinating a subject against Cryptosporidiosis. The method also provides the step of providing said subject with one or more sporozoite antigens or one or more vectors containing nucleotides coding for said one or more sporozoite antigens. The one or more sporozoite antigens may be, for example, SRK, CP15 or profilin from Cryptosporidium.

### BRIEF DESCRIPTION OF THE DRAWINGS

FIG. 1. Aligned sequence of the SRK gene and protein.

FIG. 2. Aligned sequence of the CP15 gene and protein.

FIG. 3. Aligned sequence of the profilin gene and protein.

FIGS. 4A and B. FIGS. 4A and B: Polyacrylamide gel electrophoresis of overexpressed Cp15, Profilin, and SRK protein in bacterial vectors. A, pTriEx-4 for Cp15 and Profilin, pET44 for SRK. B, pSEC10 ClyA fusions of all proteins.

FIGS. 5A and B. Analysis of humoral immune response against SRK and Cp15 in adult mice. Measurement of total IgG titer by ELISA after immunization with A, Cp15 and B, SRK.

FIG. 6A-C. Humoral and cellular immune response of mice to SRK. A, immunoglobulin A; B, lymphoproliferation; C, gamma interferon (INF).

FIG. 7A-C. Humoral and cellular immune response of mice to CP15. A, immunoglobulin A; B, lymphoproliferation; C, gamma interferon (INF).

FIG. 8A-B. Humoral and cellular immune response of mice to profilin. A, immunoglobulin A; B, lymphoproliferation.

FIG. 9. Analysis of protection against cryptosporidiosis of RAG−/− mice after adaptive transfer of Cp15 and SRK activated spleen cells from normal mice.

Assessment of oocyst numbers in stool samples from RAG−/− challenged with 1×10⁶ Cryptosporidium parvum oocysts after adoptive transfer of spleen cells from adult C57BL/6 mice immunized with Cp15 and SRK compared to the control. C57BL/6 mice were immunized with live Salmonella expressing ClyA fusions with Cp15 and SRK, or ClyA alone (control), and boosted twice in 2 weeks intervals with purified recombinant proteins Cp15 and SRK, or with PBS (control). At day 42 spleens were removed and splenocytes were isolated and transferred in RAG−/− mice.

FIG. 10A-C. Analysis of protection against cryptosporidiosis induced by Cp15 in neonate mice. A. Measurement of weight gain of neonate mice challenged with Cryptosporidium parvum after single dose immunization with ClyA fusion constructs of SRK and Cp15, or ClyA alone delivered via Salmonella live vector. PBS served as an additional control. B. Assessment of oocyst shedding. Measurement of oocyst numbers per mg of stool from neonatal mice immunized with Salmonella expressing ClyA alone (pSEC10) or ClyA in fusion with Cp15 by qRT-PCR at 20-29 and 30-39 days of age. C. Cytokine pattern of neonate mice after immunization with a single dose of Salmonella expressing ClyA (pSEC10) alone or in fusion with Cp15. Samples were collected prior to challenge and analyzed by qRT-PCR.

FIG. 11A-B. Sequence of plasmid pTriEx-4/60194 containing sequences encoding SRK. A, DNA (plasmid sequence in small font, His+S tag sequences in capitals and underlined, SRK sequence in capitals); B, amino acid sequence of SRK as coded by plasmid (His+S tag underlined).

FIG. 12A-B. Sequence of plasmid pTriEx-4/60368 containing sequences encoding Cp15. A, DNA (plasmid sequence in small font, His+S tag sequences in capitals and underlined, SRK sequence in capitals); B, amino acid sequence of SRK as coded by plasmid (His+S tag underlined).

FIG. 13A-B. Sequence of plasmid pTriEx-4/30189 containing sequences encoding (Profilin). A, DNA (plasmid sequence in small font, His+S tag sequences in capitals and underlined, SRK sequence in capitals); B, amino acid sequence of SRK as coded by plasmid (His+S tag underlined).

### DETAILED DESCRIPTION

The vaccines of the invention comprise one or more of three sporozoite-expressed Cryptosporidium hominis antigens. Exemplary antigens include SRK, CP15 and profilin. Contrary to conventional wisdom, these antigens were selected in part because they are not immunodominant, and thus have not been subjected to strong selection selective pressure exerted by the host immune system. Due to the similarity of the primary, secondary and tertiary structure of these antigens among various Cryptosporidium species, the vaccines of the invention provide protection against Cryptosporidiosis caused by any of several Cryptosporidium species that infect both humans and non-human mammals. The vaccines are beneficial on two fronts: 1) for the direct immunization of humans and other mammals for the sake of protecting the immunized subject from disease symptoms; and 2) to stop or minimize the shedding of oocysts into the environment, thereby decreasing the number of infectious agents to which a naïve subject is likely to be exposed, and stopping or slowing the spread of infection. Significantly, in the delivery system developed, the SRK, CP15 and profilin antigens elicit a strong long-term adaptive immune response against Cryptosporidium in vaccine recipients. In particular, mucosal immune responses are elicited in the intestinal mucosa. This is important because the site of Cryptosporidium infection is the intestinal epithelium.

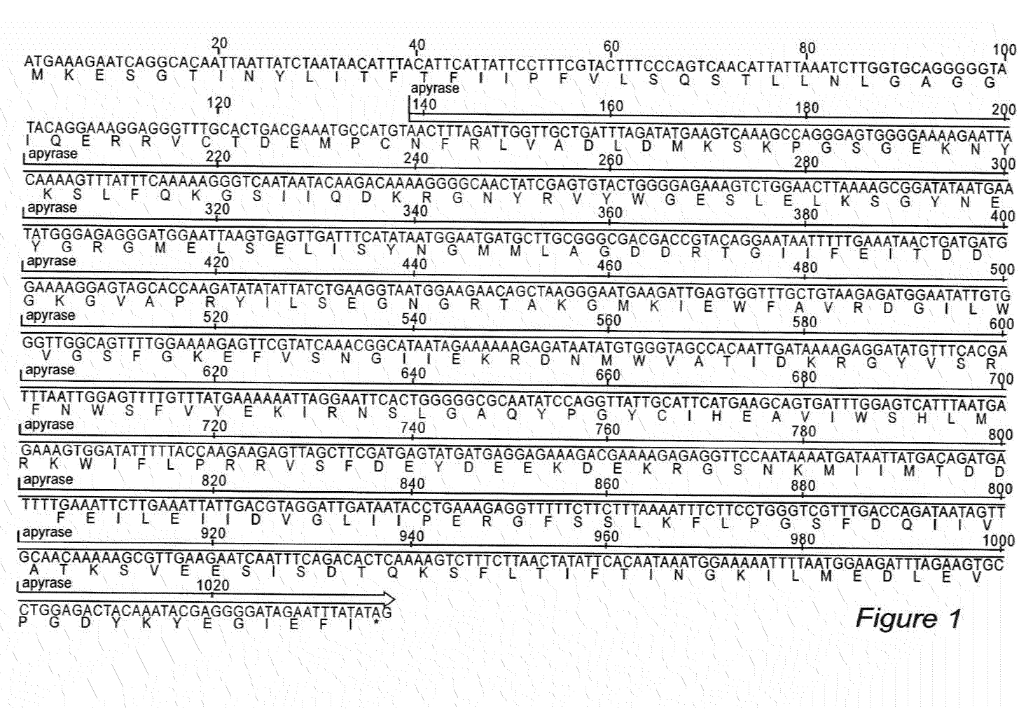

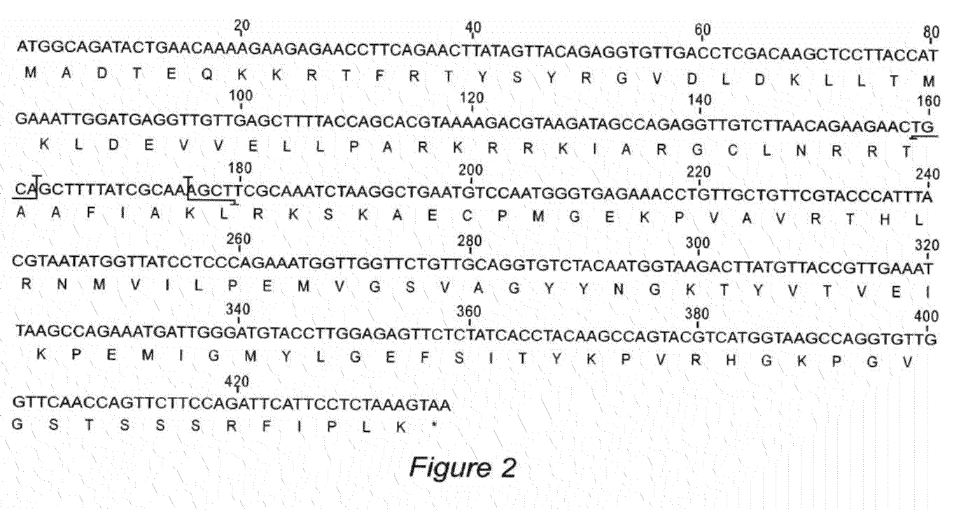

The Cryptosporidium hominis “similar to riken” (SRK) antigen shows significant promise as a potential target for a vaccine against the disease caused by the parasite in humans and other mammals, e.g. agriculturally important animals. The amino acid (SEQ ID NO: 1) and gene sequence (SEQ ID NO: 2) of SRK are presented in FIG. 1. The SRK gene is 1038 bases long, and encodes a protein of 345 amino acids. The gene has an N-terminal signal peptide with a putative cleavage site at amino acids 22-23, 1 transmembrane domain (estimated length 20 amino acids) and no glycosylphosphatidylinositol (GPI) signal anchor signal, suggesting that this protein is likely to be secreted. SRK has been immunolocalized to the apical complex of Cryptosporidium sporozoites. Annotation of the Cryptosporidium genome revealed suggested, and we have confirmed, that SRK is an apyrase (apyrase domain located at position 47 to 345), an enzyme that degrades nucleotides to nucleosides. Cryptosporidium lacks the ability to synthesize nucleotides, suggesting that this enzyme may play a critical role in the interaction of the parasite with the host cell, and may be essential. This putative essential role of SRK could enhance its efficacy as a vaccine target, or as a target for chemotherapy. For example, a compound or drug (e.g. a small molecule or “designer” drug) that inhibits (preferably specifically or selectively inhibits) the activity of SRK may be provided or administered to a subject that is infected with or suspected of being infected with Cryptosporidium. Such a drug would kill the parasite, and/or slow its development and/or interrupt its life cycle within the host subject, and/or inhibit or impede its transmission to another host. Such drugs might also be administered therapeutically to treat an existing infection, or prophylactically, e.g. to uninfected subjects prior to travel to an area where Cryptosporidosis is endemic or where Cryptosporidium parasites are likely to be encountered, or to animals that are exposed to or subject to infection. Cp15 was initially identified by Jenkins and Fayer (6) as an antigen that induced an immune response in infected mammals. These investigators cloned a partial gene encoding Cp15, but did not pursue the gene/protein as a potential vaccinogen. Other proteins with a molecular size of ˜15 kDa have also been described as Cp15, and these genes/proteins have been used as an antigen either as a protein or DNA vaccine, eliciting both humoral and cellular immune responses (c.f., ref 7 and 8). However, these latter genes/proteins are not related to the Cp15 described herein. The Cryptosporidium hominis Cp15 antigen, the amino acid (SEQ ID NO: 3) and gene (SEQ ID NO: 4) sequence of which is presented in FIG. 2, shows significant promise as a potential target for a vaccine against the disease caused by the parasite in both humans and non-human mammals. The Cp15 gene is 438 bases long, and encodes a protein of 145 amino acids with a ribosomal protein conserved domain and no signal peptide sequence. This antigen has proven to be an efficacious vaccine target.

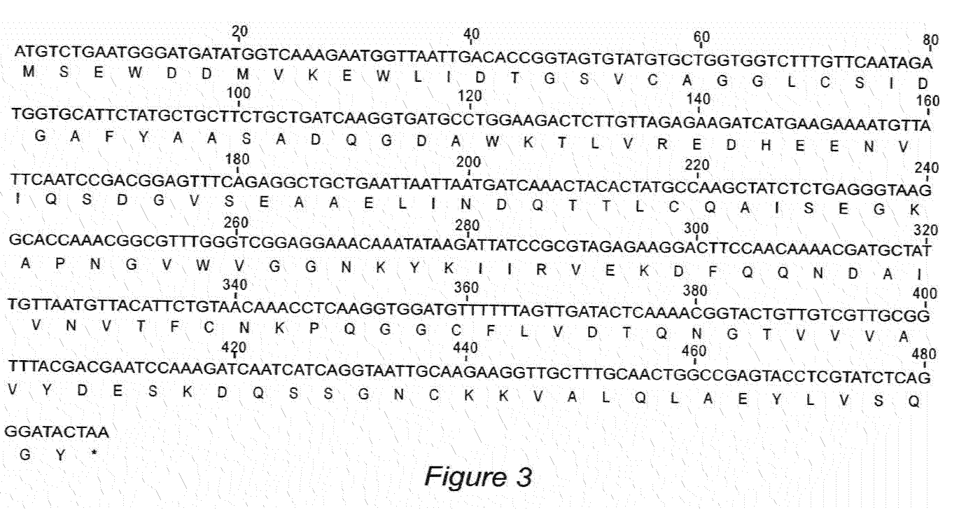

A Cryptosporidium protein resembling Profilin, which in other organisms is has been reported to be a component of the cytoskeleton, is reported here as a potential vaccinogen. In other organisms, profilin has been shown to induce a strong innate immune response in animal models through interaction with the toll like receptor 11—TLR 11 (10). However, the protein has not been anticipated as a vaccine candidate because of the lack of TLR 11 in humans (see below), and the ability of the protein to induce a strong innate immune response has not been explored previously. The amino acid (SEQ ID NO: 5) and gene (SEQ ID NO: 6) sequence of the Cryptosporidium hominis profilin gene are presented in FIG. 3. The profilin gene is 486 bases, and encodes a protein of 162 amino acids. The gene has a no signal peptide but has a predicted site for a GPI anchor at position 142. While the mammalian receptor of profilin—TLR 11—is expressed in most animal models, including mice, cattle, and pigs, the human TLR 11 gene in humans is an inactive pseudogene (10). Thus, profilin is likely to play an important role in the control of endemic cryptosporidiosis in animal populations. Such animals include both agriculturally important animals, (e.g. calves, pigs, etc.) and domestic animals (dogs, cats, etc). Protection of animals has two purposes: 1) the health, well-being, and economic viability of the animals themselves; and 2) prevention of the spread of Cryptosporidium by the animals to recreational and potable water supplies, which causes human infections.

The vaccine preparations of the invention preferably comprise one or more antigens that are expressed in sporozoites of Cryptosporidium. Examples of such antigens include but are not limited to the three antigens SRK, CP15 and profilin. By “antigens that are expressed in sporozoites” we mean antigens that are expressed largely or exclusively during the sporozoite stage of the Cryptosporidium life cycle. Such antigens may be originally identified in Cryptosporidium hominis, but this need not always be the case, as other species of also exhibit the sporozoite life cycle stage. In particular, the antigens SRK, CP15 and profilin from other Cryptosporidium species may be utilized in the present invention. The vaccine preparations may contain of single antigen, or cocktails or mixtures of two or three antigens may be used. The vaccines may comprise the antigens as proteins or segments thereof, or, alternatively, the vaccines are nucleic acid vaccines that comprise nucleic acid sequences that encode the proteins or partial proteins. As such, the invention provides the proteins or antigenic portions thereof, and nucleic acid sequences that encode the proteins or antigenic portions thereof, and recombinant hosts, in combination with other suitable vaccine components. In addition, the vaccines may be a bacterium transformed to include one or more of the nucleic acid sequences, where the antigens are expressed from the nucleic acid sequence.

With respect to the nucleic acid sequences encoding SRK, CP15 and profilin disclosed herein (SEQ ID NOS: 2, 4 and 6), those of skill in the art will recognize that many variants (derivatives) of the sequences may exist or be constructed which would be suitable for use in the practice of the present invention. For example, with respect to the translation of amino acid sequences from the nucleic acid sequences, due to the redundancy of the genetic code, more than one codon may be used to encode an amino acid. Further, as described below, changes in the amino acid primary sequence may be desired, and this would necessitate changes in the encoding nucleic acid sequences. In addition, those of skill in the art will recognize that many variations of the nucleic acid sequences may be constructed for purposes related to other aspects of the invention, for example: for cloning strategies (e.g. the introduction of restriction enzyme cleavage sites for ease of manipulation of a sequence for insertion into a vector, for rendering the sequence compatible with the cloning system vector or host, for enabling fluorescent or affinity labeling technologies, etc.), for purposes of modifying transcription (e.g. the introduction of specific promoter or enhancer sequences, insertion or deletion of splice signals, for enhancing or negatively regulating transcription levels, for regulating polyadenylation, for controlling termination, and the like), or for modification of active or inactive domains, for elimination or modification of certain activities or domains, for optimizing expression due to codon usage or other compositional biases, for addition of immunologically relevant (enhancing or inhibiting) sequences, or for any other suitable purpose. All such variants of the nucleic acid sequences disclosed herein are intended to be encompassed by the present invention, provided the sequences display homology in the range of about 50 to 100%, and preferably about 60 to100%, or more preferably about 70 to 100%, or even more preferably about 80 to 100%, or most preferably about 90 to100%, i.e. about 95, 96, 97, 98, 99 or 100% homology to the disclosed sequences. The homology is with reference to the portion of the nucleic acid sequence that corresponds to the sequence disclosed herein, and is not intended to cover additional elements such as promoters, vector-derived sequences, restriction enzyme cleavage sites, etc. Those of skill in the art are well acquainted with methods to determine nucleic acid similarity or homology using software alignment tools such as FASTA, the BLAST suite of programs, CLUSTAW, Lineup, Pileup (GCG), or many others. All such variants or derivatives of the nucleic acid sequences disclosed herein are intended to be encompassed by the invention.

In addition, the nucleic acids of the present invention are not limited to DNA or cDNA, but are intended to encompass other nucleic acids as well, such as RNA (e.g. mRNA, RNA-DNA hybrids, etc.) and various modified forms of DNA and RNA known to those of skill in the art. For example, for use in vivo, nucleic acids may be modified to resist degradation via structural modification (e.g. by the introduction of secondary structures, such as stem loops, or via phosphate backbone modifications, etc.). Alternatively, the nucleic acids may include phosphothioate or phosphodithioate rather than phosphodiesterase linkages within the backbone of the molecule, or methylphosphorothiate terminal linkages. Other variations include but are not limited to: nontraditional bases such as inosine and queosine; acetyl-, thio- and similarly modified forms of adenine, cytidine, guanine, thymine and uridine; stabilized nucleic acid molecules such as nonionic DNA analogs, alkyl- and aryl phosphonates; nucleic acid molecules which contain a diol, such as tetrahyleneglycol or hexaethyleneglycol, at either or both termini; etc. Further, the nucleic acid molecules may be either single or double stranded, or may comprise segments of both single and double strand nucleic acid. All such variants or derivatives of the nucleic acid sequences disclosed herein are intended to be encompassed by the invention.

With respect to the antigens themselves, either the full length antigens, or antigenic sequences from within the full length sequence (e.g. antigenic determinants), may be used to elicit an immune response, either by administering the antigen directly, or by administering a nucleic acid that encodes the antigen. “Antigen” may refer to a full-length sequence as set forth, for example, in SEQ ID NOS: 1, 3 and 5, or a peptide or polypeptide that encompasses one or more antigenic regions of those sequences. By “antigenic region” we mean a section of the sequence that elicits an immune response that is at least about 50, preferably at least about 60, more preferably at least about 70, most preferably at least about 80% (e.g. 85, 90, 95 or even 100%) of that of the full length sequence. Such regions may be peptides, polypeptides, or proteins. In general, for the purposes of the present invention, a peptide comprises about 15 or fewer amino acids, a polypeptide comprises from about 15 to about 100 amino acids, and a protein comprises about 100 or more amino acids, although the terms may be used interchangeably herein. The antigenic peptides, polypeptides and proteins of the invention are generally provided as recombinant molecules, although the amino acid sequences may also be produced synthetically via known peptide synthesis techniques. Generally, for inclusion in the preparations of the invention, a recombinant antigen will be substantially pure, i.e. largely (e.g. at least about 70%, and preferably at least about 80%, and more preferably at least about 90-95% or more) free of other molecules or substances that are generally considered to be “contaminants” (e.g. other proteins, nucleic acids, lipids, cellular debris, etc.).

The invention also encompasses variants (derivatives) of the antigens. For example, variants may exist or be constructed which display: conservative amino acid substitutions; non-conservative amino acid substitutions; truncation by, for example, deletion of amino acids at the amino or carboxy terminus (e.g. deletion of signal sequences), or internally within the molecule; or by addition of amino acids at the amino or carboxy terminus, or internally within the molecule, for example: the addition of a histidine or similar tag for purposes of facilitating protein isolation or expression; the substitution of residues to alter solubility properties, usually to increase solubility; the replacement of residues which comprise protease cleavage sites to eliminate proteolysis and increase in vivo stability; the replacement of residues to form a convenient protease cleavage site; the addition or elimination of glycosylation sites; modifications to facilitate expression in an expression system of interest such as Pichia, baculovirus, mammalian expression systems, etc.; and the like, for any reason. Such variants may be naturally occurring (e.g. as the result of natural variations between species or between individuals, or as a result of different expression systems used to produce the amino acid sequence, etc.); or they may be purposefully introduced (e.g. in a laboratory setting using genetic engineering techniques). The amino acid sequences may be in a variety of forms, including neutral (uncharged) forms, or forms which are salts, and may contain modifications such as glycosylation, side chain oxidation or deamidation, phosphorylation and the like. Also included are amino acid sequences modified by additional substituents such as glycosyl units, lipids, or inorganic ions such as phosphates, as well as modifications relating to chemical conversions or the chains, such as oxidation of sulthydryl groups.

All such variants of the sequences disclosed herein are intended to be encompassed by the teachings of the present invention, provided the variant protein/polypeptide displays sufficient identity to the original sequences, the original sequence being a sequence as disclosed herein, or an amino acid sequence that can be translated from a nucleic acid sequence disclosed herein. Preferably, amino acid identity will be in the range of about 50 to 100%, and preferably about 60 to100%, or more preferably about 70 to 100%, or even more preferably about 80 to 100%, or most preferably about 90 to 100%, or even 95 to 100%, i.e. 95, 96, 97, 98, 99 or 100%, of the disclosed sequences. The identity is with reference to the portion of the amino acid sequence that corresponds to the original amino acid sequence as translated directly from the nucleic acid sequences disclosed herein, i.e. not including additional elements that might be added, such as sequences added to form chimeric proteins, histidine tags, linker sequences, etc. Those of skill in the art are well acquainted with the methods available for determining the identity between amino acid sequences, for example, FASTA, FASTP, the BLAST suite of comparison software, ClustalW, Lineup, Pileup, or many other alignment software packages.

Particular variants of interest include but are not limited to: an SRK variant that includes only amino acids 23-345 (i.e. without the N-terminal signal sequence) or that includes only amino acids 47-345, the putative conserved apyrase domain

The invention also provides vectors comprising nucleic acid sequences engineered or genetically engineered to encode and express one or more of SRK, CP15 and/or profilin, or antigenic portions thereof as described herein. Those of skill in the art are well-acquainted with various vectors that may be used e.g. for manipulation of nucleic acid sequences during genetic engineering procedures, for storage of stocks of the nucleic acids, for expression of an amino acid sequence encoded by the nucleic acid, for expression in bacterial, fungal, insect or other host systems, for delivery of DNA vaccines, for amplification of the DNA, for sequence analysis, for molecular interaction studies, etc. Many such vectors are known to those of skill in the art, and include but are not limited to plasmids, adenoviral vectors, various expression vectors e.g. pTriEX4, pET41, pET44, and others of the pET series; the pUC vector series; the BlueScript series, derivatives of pBR322 with ColE1 origin of replication; the TOPO vector series; the Gateway vectors; the TET repressor vectors; BAC vectors [pBeloBACs, pCC1BAC, etc.]; pcDNA301 and related plasmids with the CMV promoter; pBAC insect vectors; pIEX for insect cells, various bacterial (e.g. Escherichia coli) or probiotic-based (e.g. Lactobacillus) expression vectors; vectors for use in Pichia, and yeast expression systems, and many others. Of special interest in the present invention are the expression vectors pTriEX4, pET41, pET44, and pSEC10.

As used herein, the term “vector” also includes whole organisms (e.g. also referred to as “hosts”) that may be genetically engineered to encode and express one or more of the antigens described herein. Such vaccine expression vectors are of special interest in the present invention, particularly those that are suited for administration of nucleic acid-based vaccines in humans and other mammals. For example, various attenuated live vaccine expression vectors (bacterial or viral) may be utilized, including but not limited to Salmonella vectors (e.g. pSEC 10 ClyA and related vectors); various attenuated Mycobacterial vectors; various viral vectors such as adenoviral, influenza, etc.; and similar Lactobacillus vectors, etc

In some embodiments, vectors containing nucleic acid sequences that encode the antigens of the invention will encode a single antigen. However, this need not always be the case. Such vectors may contain sequences encoding more than one antigen of the invention, either as separate, discrete sequences, or combined into a single chimeric sequence. For example, in the case of an expression vector, one, two or all three nucleic acids encoding SRK, SP15 and profilin may be present in the vector. The nucleic acids may be expressed separately, resulting in the translation of one antigen for each nucleic acid, or, alternatively, a single polypeptide chain containing more than one antigen (e.g. translated in tandem) may be produced. For example, one or more antigens (or antigenic regions) may be expressed from a single contiguous nucleic acid sequence as a chimera or fusion protein. Alternatively, the amino acid sequences of the invention may be expressed as part of a chimeric or fusion protein comprising amino acid sequences from another source, e.g. antigenic sequences known to be useful as adjuvants (e.g. PADRE [and other Pan-DR T helper cell epitope], hepatitis B core antigen, DNA sequences like CPG oligonucleotides or other Immunomodulatory oligonucleotides (IMGs), other chemokines, CTB or cholera toxin B subunit, Ricin B and other plant toxin subunits, LPS or lipopolysaccharide, KLH [key hole limpet hemocyanin], sequences that permit targeting of the protein to a specific location (e.g. to the small intestines), etc. The invention also comprehends a cell or cells containing one or more or such vectors, and the vectors may be the same or different. Further, the cells may be either in vitro or in vivo.

The invention also provides antibodies directed to the amino acid sequences of SRK, CP15 and/or profilin. As used herein, the term “antibody” refers to a polypeptide or group of polypeptides composed of at least one antibody combining site. An “antibody combining site” is the three-dimensional binding space with an internal surface shape and charge distribution complementary to the features of an epitope of an antigen, which allows binding of the antibody with the antigen. “Antibody” includes, for example, vertebrate antibodies, hybrid antibodies, chimeric antibodies, humanized antibodies, altered antibodies, univalent antibodies, Fab proteins and fragments, and single domain antibodies. Antibodies to the proteins of the invention, both polyclonal and monoclonal, may be prepared by conventional methods that are well-known to those of skill in the art. If desired, the antibodies (whether polyclonal or monoclonal) may also be labeled using conventional techniques.

Such antibodies may be used, for example, for affinity chromatography, immunoassays, and for distinguishing or identifying Cryptosporidium proteins or portions thereof. In a preferred embodiment of the invention, such antibodies may be used therapeutically, e.g. for administration to patients suffering from cryptosporidiosis, or prophylactically in order to prevent cryptosporidiosis in patients at risk for developing the disease.

The present invention provides compositions for use in eliciting an immune response and/or for vaccinating an individual against Cryptosporidium. The compositions include one or more substantially purified SRK, CP15 and/or profilin antigens as described herein, or nucleic acid sequences encoding such antigens, and a pharmacologically suitable/compatible carrier. The preparation of such compositions for use as vaccines is well known to those of skill in the art. Typically, such compositions are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. In addition, the composition may contain other adjuvants. If it is desired to administer an oral form of the composition, various thickeners, flavorings, diluents, emulsifiers, dispersing aids or binders and the like may be added. The composition of the present invention may contain any such additional ingredients so as to provide the composition in a form suitable for administration. The final amount of antigen or encoding nucleic acid in the formulations may vary. However, in general, the amount in the formulations will be from about 1-99%.

The invention also encompasses methods of administering compositions comprising the antigens. The antigen compositions (preparations) of the present invention may be administered by any of the many suitable means which are well known to those of skill in the art, including but not limited to by injection, inhalation, orally, intravaginally, intranasally, by ingestion of a food or probiotic product containing the antigen, topically, as eye drops, via sprays, etc. In preferred embodiments, the mode of administration is by injection or intranasally (e.g. expressed from Salmonella). When used in this manner, one of skill in the art will recognize that the Salmonella are not ‘host bacteria’ per se, but do colonize the subject to whom they are administered, albeit briefly. E. coli, and other bacteria may also be employed. The advantage of this is that the bacteria deliver the antigen to the nasopharyngeal mucosa (and also could be swallowed and deliver the antigen to the gastrointestinal mucosa), thus inducing an appropriate mucosal immune response. In addition, the compositions may be administered in conjunction with other treatment modalities such as substances that boost the immune system, various chemotherapeutic agents (e.g. nitazoxanide), other antigens, and the like. In a preferred embodiment, the vaccinogen is delivered intranasally, or in a combined regimen including: 1) intranasal inoculation of the live vector vaccine expressing a fusion protein followed by 2). intraperitoneal (IP), intraomuscular (IM) or other administration of the purified recombinant protein. Intranasal inoculation induces the mucosal immunity that is desired against cryptosporidiosis. Those of skill in the art are aware of such prime-boost regimens, and are well-acquainted with the routes of administration and planning the time intervals between the prime and boost administrations. Further, more than one boost may be administered.

The compositions of the invention may be administered prophylactically (i.e. before exposure to the infectious agent) or therapeutically (i.e. after exposure or probable exposure to the infectious agent, or after infection by the infectious agent).

In some embodiments of the invention, the patient or subject that is vaccinated against Cryptosporidiosis is a human. In other embodiments, the subject or patient is a non-human mammal, and may be referred to herein as an “animal”. In other words, the vaccines and immunogenic preparations described herein have veterinary applications. Animals which can benefit from the administration of the vaccines of the invention include but are not limited to various agriculturally important animals such as cattle, sheep, pigs, horses, goats, domestically raised deer, bison, etc. Domestic animals that can benefit from administration of a profilin-based vaccine include but are not limited to cats, dogs, hamsters, gerbils, mice, rats, guinea pigs, birds such as parrots, etc. Horses, especially work or race horses, may also benefit from the vaccine. Further, non-domesticated animals such as those found in zoos, preserves, and various protected habitats, may also benefit. Any mammal that is susceptible to infection (either an active infection that causes disease, or an asymptomatic “carrier” infection) may benefit from being a recipient of the vaccines of the invention. In preferred embodiments of the invention, the antigens that are employed in a vaccine for humans include SRK and Cp15. In preferred embodiments of the invention, the antigens that are employed in a vaccine for non-human mammals include SRK Cp15 and Profilin.

The quantity of an individual antigen that is administered in the practice of the invention may be in the range of from about 10 to about 500 μg of protein/kg of body weight, and preferably from about 20 to about 200 μg of protein/kg of body weight, either administered directly, or via translation from an administered nucleic acid. The amount of vaccine provided may depend on the patient, the route of delivery, and the composition used to provide the nucleotide sequence or antigen.

Due to the relatively high identity amongst proteins from various Cryptosporidium species, vaccination with the antigens of the invention can provide protection against many species of the parasite, including but not limited to: C. parvum, C. hominis (previously known as C. parvum genotype 1), C. canis, C. felis, C. meleagridis, and C. muris, each of which can also cause disease in humans and other species, particularly mammals.

The present invention provides compositions for use in eliciting an immune response, preferably a long-term adaptive immune response, against Cryptosporidium. The compositions may be utilized as a vaccine against Cryptosporidium. By “eliciting an immune response” we mean that an antigen stimulates synthesis of specific antibodies at a titer of about >1 to about 1×10⁶ or greater. Preferably, the titer is from about 10,000 to about 1×10⁶ or more, as measured by, e.g. enzyme Linked Immunosorbent Assay (ELISA). Alternatively, or in addition, “eliciting an immune response” refers to an increase in cellular (e.g. T-cell) proliferation, as measured, e.g. by ³H thymidine incorporation. Alternatively, or in addition, “eliciting an immune response” refers to, for example, an increase in production of γ interferon (IFN), TNF-alpha, IL6, IL12 and other cytokines.

In some cases, vaccination with the compositions of the invention may prevent infection by Cryptosporidium, and/or prevent the manifestation of symptoms associated with Cryptosporidiosis. However, this need not be the case, as many benefits can accrue even if the symptoms are only partially relieved or attenuated. For example, “vaccine” may mean an antigen preparation that elicits an immune response that results in a decrease in parasite burden and/or a decrease in the number of parasite oocysts that are shed, of a least about 20%, preferably about 30%, more preferably about 40%, even more preferably about 50%, and most preferably about 60, 70, 80, 90 or even 100%, compared to a non-vaccinated (e.g. adjuvant alone) control subject. Those of skill in the art will recognize that the success or efficacy of a “vaccine” against a protozoan such as Cryptosporidium is not measured according to the same standards as those that are used for vaccines against other infectious agents (e.g. viruses, bacteria, etc.). The goals of administering a protozoal vaccine are different. In particular, for a protozoal vaccine to be considered “protective” or efficacious if it decreases the parasite or disease burden, or prevents transmission of the parasite or disease to other susceptible hosts.

Another important aspect of the invention is based on the discovery that the profilin antigen displays potential significant adjuvant properties. In other words, not only does this antigen elicit an immune response to its own antigenic determinants, administration of this antigen in conjunction with other antigens (which may or may not be Cryptosporidium antigens) increases the observed immune response of the recipient to those other antigens. For example, state of the art adjuvants activate innate immunity to elicit strong immune responses directed to specific antigens. Since Profilin activates macrophages via TLR-11, and thereby the innate immune response, it is reasonable to expect that it can play a major role as an adjuvant when administered with other antigens, whether from Cryptosporidium or other pathogens. Thus, the profilin antigen may be used in any vaccine system or vaccination protocol to increase or augment the immune response of a vaccine recipient. In a preferred embodiment, profilin is used in vaccine preparations for animals, including vaccines against Cryptosporidium or any other infectious agent (virus, bacteria, parasite, or worm).

The spirit and scope of the invention is further illustrated in the following Examples, which serve to illustrate the invention, but are not intended to limit it in any way.

### Examples

The following examples describe experiments that were carried out to test the ability of SRK, CP15 and profilin antigens to induce immunity using a mammalian mouse model system. While the results were remarkable in that robust positive responses (both humoral and cellular) were observed for all three antigens.

### Example 1

### Experiments Involving Antigen SRK

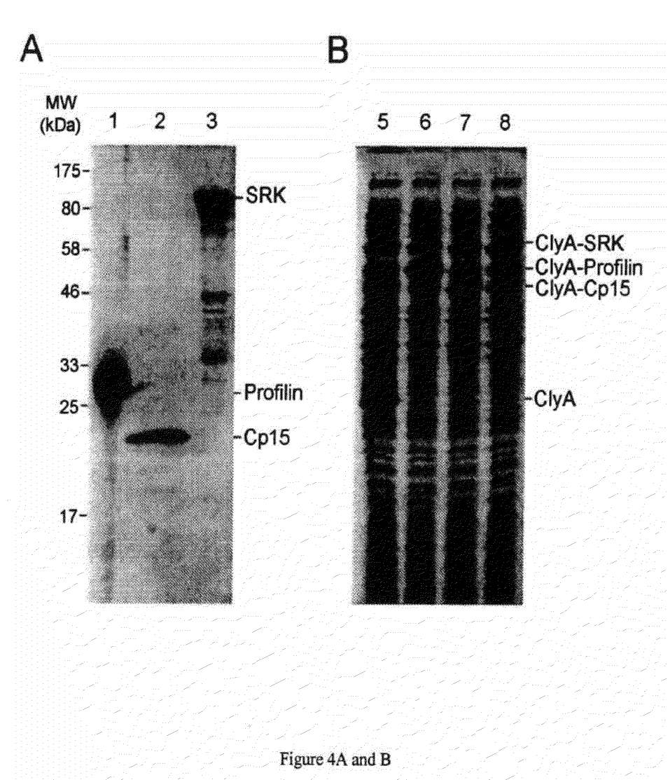

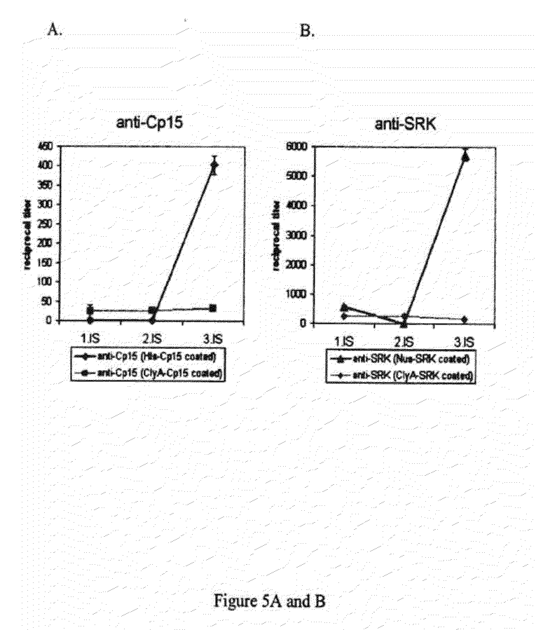

SRK expressed in bacterial expression systems. The SRK gene was ligated into pTriEX4 (His-Tag), pET41 (GST-Tag), and pET44 (Nus-Tag) Escherichia coli (E. coli) expression vectors and pSEC10 ClyA Salmonella live vaccine vector (Galen J. E., Zhao L., Chinchilla M., Wang J. Y., Pasetti M. F., Green J., et al. (2004) Adaptation of the endogenous Salmonella enterica serovar Typhi clyA-encoded hemolysin for antigen export enhances the immunogenicity of anthrax protective antigen domain 4 expressed by the attenuated live-vector vaccine strain CVD 908-htrA. Infect. Immun. 72, 7096-7106). and expressed using standard protocols. The results of these overexpression experiments are summarized in Table 1, and polyacrylamide gel electrophoresis results are presented in FIGS. 4A and B. As can be seen, SRK is expressed strongly in pTriEX4 (FIG. 4A, lane 3) and pSEC10 ClyA (FIG. 4B, lane 8).

TABLE 1

| | | pTriEX4 | pET41 | pET44 | pSEC10 |
|---|---|---|---|---|---|
| MW | Func- | (His-Tag) | (GST-Tag) | (Nus-Tag) | ClyA |
| (kDa | tion | cloned | cloned | cloned | Salmonella |
| | | expressed | expressed | expressed | |
| 37 | Un- | + Insoluble | + Insoluble | + Insoluble | + |
| | known | | | | |

These bacterial expression systems permit large scale production of SRK protein, and the pSEC10 ClyA fusion vector permits delivery of the antigen in a live secreting bacterial vector intranasally in animal models.

Immunization Assays.

Interferon gamma knockdown murine model. This model permits infection of adult mice providing much the same symptoms that are seen in human infections. Tests of the SRK protein in this model were performed. Pools of recombinant antigens were used to immunize C57B/6 adult mice in several doses given intramuscularly. Anti IFN gamma monoclonal antibody was administered after the final boost, and the mice were challenged with Cryptosporidium. All mice demonstrated a strong humoral immune response against all of the antigens administered, including SRK (FIG. 5B). In contrast, no immune response was detected against any of the candidate antigens in control mice given extracts of Cyrptosporidium (not shown).

Since the Cryptosporidium extracts did induce a humoral response, these results suggest that the selected target antigens, including SRK, are in fact not immunodominant, thus validating a key component of this project.

Antibodies immunolocalized SRK to membrane and block invasion in tissue culture. These antibodies were used to block invasion of cultured human intestinal epithelial cells (HCT8 cell line) by Cryptosporidium and tested for ability to block invasion of cultured HCT-8 cells. The antibodies showed significant inhibitory effects on the rate of infection (not shown). Antibodies were used for immunolocalization and the results confirmed that the proteins are located on or near the cell membrane, and localized to the apical complex (not shown).

Immunization protocols. Immunization protocols using DNA vaccine vectors, recombinant protein, or the intranasal delivery of live vector Salmonella ClyA fusion proteins have been tested, and an efficient protocol involving a combination of intranasal delivery of live vector vaccine and IP delivery of a recombinant protein as a boost was developed. Thus, we tested each of these antigens in adult mice to characterize the immune response (IR) to each. A protocol in which animals primed (at day 0) with live Salmonella vector (ClyA) expressing Cryptosporidium antigens, at day 14 animals were boosted with either recombinant protein or ClyA expressing Cryptosporidium proteins, and finally at day 28 animals received a second boost with recombinant protein. The results of this immunization protocol showed that all the proteins, including SRK, induced a strong antibody response, with antibody titers increasing dramatically after the first boost. Again, serum from animals immunized with sporozoite extract does not recognize the recombinant protein in dot blot assay, further supporting the above suggestion that these proteins are not immunodominant.

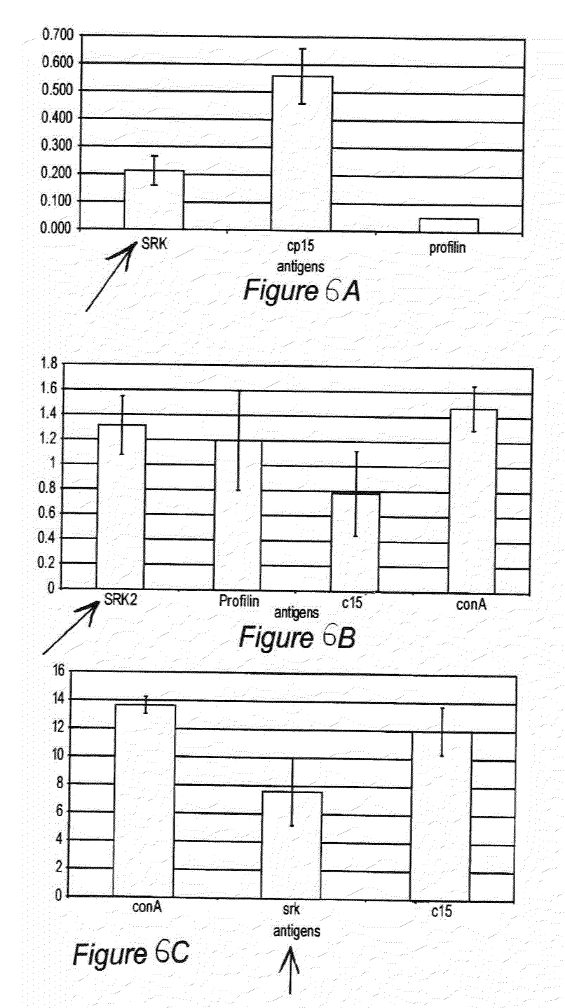

The humoral and cellular immune responses of mice to SRK. Analysis of the immunoglobulin isotypes induced by Cryptosporidium immunogens revealed that each antigen induces a unique and distinct pattern, suggesting the possibility of the induction of different T cell populations by these antigens. Significantly, profilin, SRK, and Cp15 induced production of IgA in the intestine as detected by ELISA assay (FIG. 6A, arrow shows SRK). The ability to induce a specific T cell response was also evaluated using a classical lymphoproliferation assay. Profilin, SRK and Cp15 each induced a strong proliferative response with high production of gamma-IFN, a critical cytokine for the control of the disease (see FIGS. 6B-C, where the arrows show SRK). The abilities of these antigens to protect neonatal mice from infections were examined. Each of the above antigens was delivered to 6 day old neonatal mice by intranasal administration of the live Salmonella expressing ClyA fusion proteins. Four days later, the mice were infected with Cryptosporidium oocysts. The infections were followed by quantification of the parasite by qRT-PCR, weight gain, and other characteristics.

The results showed that SRK is able to partially protect these animals from the adverse affects of Cryptosporidium infection (not shown). In particular, the SRK vaccine regimen significantly reduced oocyst shedding after challenge, despite not affecting the overall weight gain of the infant mice. This partial protection is remarkable in that a standard immunization protocol usually requires several weeks for optimal induction of immunity, whereas these results were observed after only a single dose of the antigen in the Salmonella live vaccine delivery system 48 hrs before the challenge.

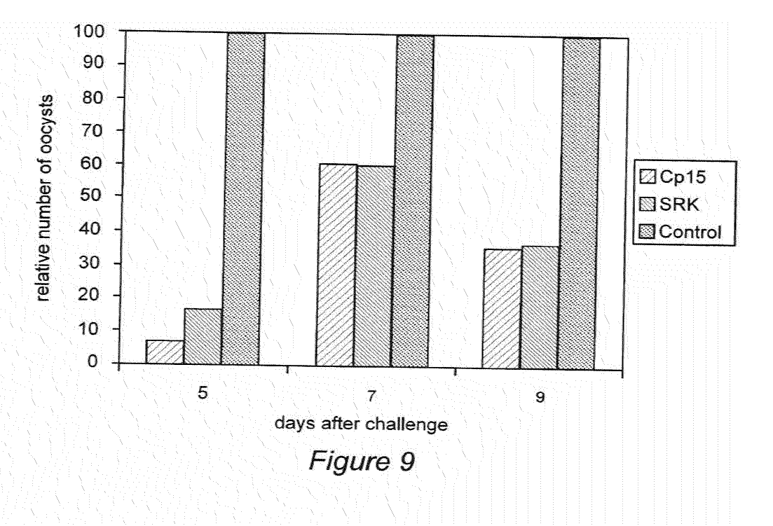

Cp15 and SRK also passively protected against infection in the RAG-2^{-/-} mouse model for Cryptosporidium. The Rag-2^{-/} mouse is defective in immunity and is unable to mount either a humoral or cellular immune response. Passive transfer experiments using spleen cells derived from animals immunized with Cp15 and SRK showed a significant reduction of the oocyst shedding in RAG-2^{-/-} mice that received SRK and Cp15 when compared to recipients of spleen cells from animals immunized with PBS (see FIG. 9). Thus, this experiment demonstrates that a strong adaptive immune response will protect susceptible individuals from disease and likely reduce shedding of the parasite.

### Example 2

### Experiments Involving Antigen CP15

Cp15 expressed in bacterial expression systems. The Cp15 gene was ligated into pTriEX4 (His-Tag), pET41 (GST-Tag), and pET44 (Nus-Tag) E. coli expression vectors and pSEC10 ClyA Salmonella live vaccine vector as described above and expressed using standard protocols. The results of these overexpression experiments are summarized in Table 2, and polyacrylamide gel electrophoresis results are presented in FIGS. 4A and B. As can be seen, CP15 is expressed very well in pTriEX4 (FIG. 4A, lane 2) and pSEC10 ClyA (FIG. 4B, lane 7).

TABLE 2

| | | pTriEX4 | pET41 | pET44 | pSEC10 |
|---|---|---|---|---|---|
| MW | Func- | (His-Tag) | (GST-Tag) | (Nus-Tag) | ClyA |
| (kDa | tion | cloned | cloned | cloned | Salmonella |
| | | expressed | expressed | expressed | |
| 17 | Attach- | + Insoluble | + Insoluble | + Soluble | + |
| | ment | | | | |

Antibodies immunolocalize Cp15 to membrane and block invasion in tissue culture. These antibodies were used to block invasion of cultured human intestinal epithelial cells (HCT8 cell line) by Cryptosporidium. The antibodies showed significant inhibitory effects on the rate of infection (not shown). Antibodies were used for immunolocalization and the results confirmed that the proteins are located on or near the cell membrane, and localized to the apical complex (not shown).

Immunization protocols. Immunization protocols using DNA vaccine vectors, recombinant protein, or the intranasal delivery of live vector Salmonella ClyA fusion proteins have been tested, and an efficient protocol involving a combination of intranasal delivery of live vector vaccine and IP delivery of a recombinant protein as a boost was developed. Thus, we tested each of these antigens in adult mice to characterize the IR to each. A protocol in which animals primed (at day 0) with live Salmonella vector (ClyA) expressing Cryptosporidium antigens, at day 14 animals were boosted with either recombinant protein or ClyA expressing Cryptosporidium proteins, and finally at day 28 animals received a second boost with recombinant protein. The results of this immunization protocol showed that all the proteins, including Cp15 (FIG. 5A), induced a strong antibody response, with antibody titers increasing dramatically after the first boost. Again, serum from animals immunized with sporozoite extract does not recognize the recombinant protein in dot blot assay, further supporting the above suggestion that these proteins are not immunodominant.

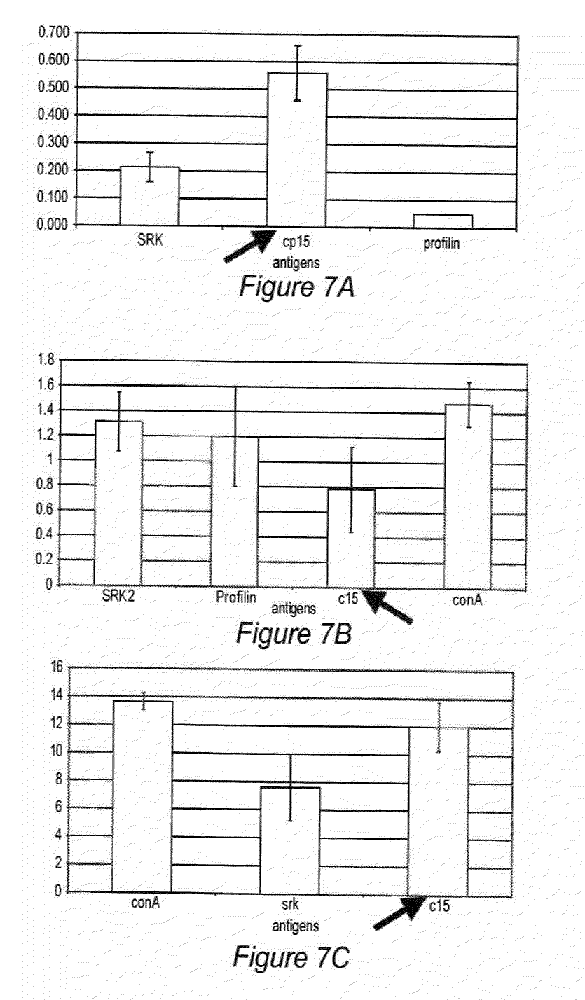

The humoral and cellular immune responses of mice to Cp15. Analysis of the immunoglobulin isotypes induced by these immunogens revealed that each antigen induces a unique and distinct pattern, suggesting the possibility of the induction of different T cell populations by these antigens. Significantly, profilin, SRK, and Cp15 induced production of IgA in the intestine as detected by ELISA assay (FIG. 7A, where the arrow shows Cp15). The ability to induce a specific T cell response was also evaluated using a classical lymphoproliferation assay. Profilin, SRK and Cp15 each induced a strong proliferative response with high production of γ-IFN, a critical cytokine for the control of the disease (FIG. 7B, where the arrow shows Cp15).

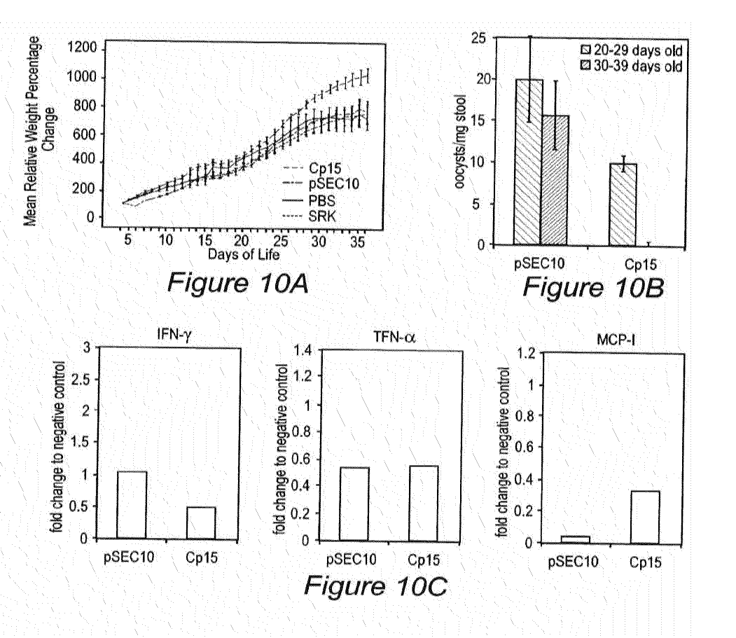

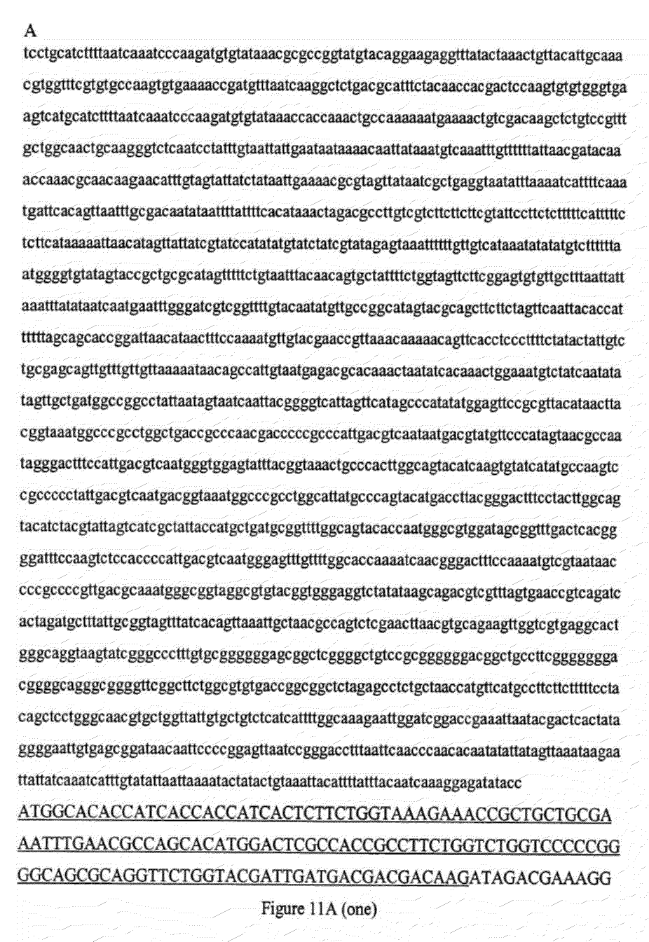

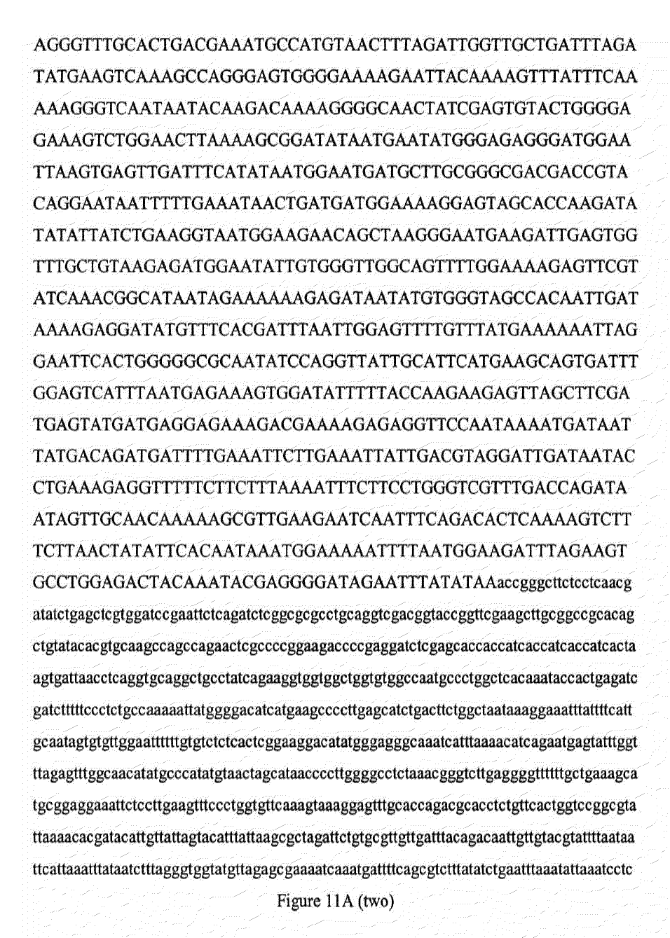

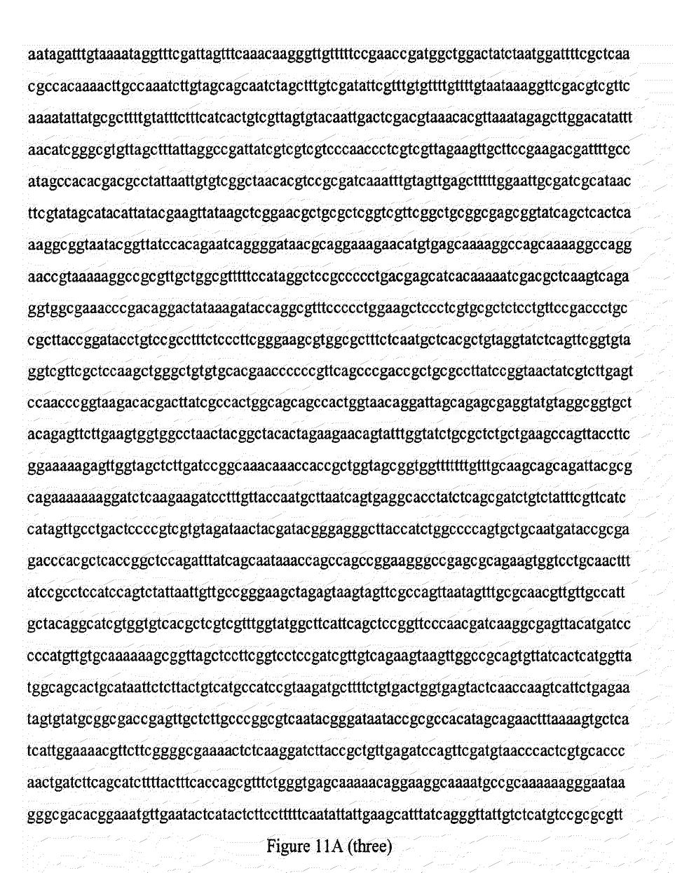

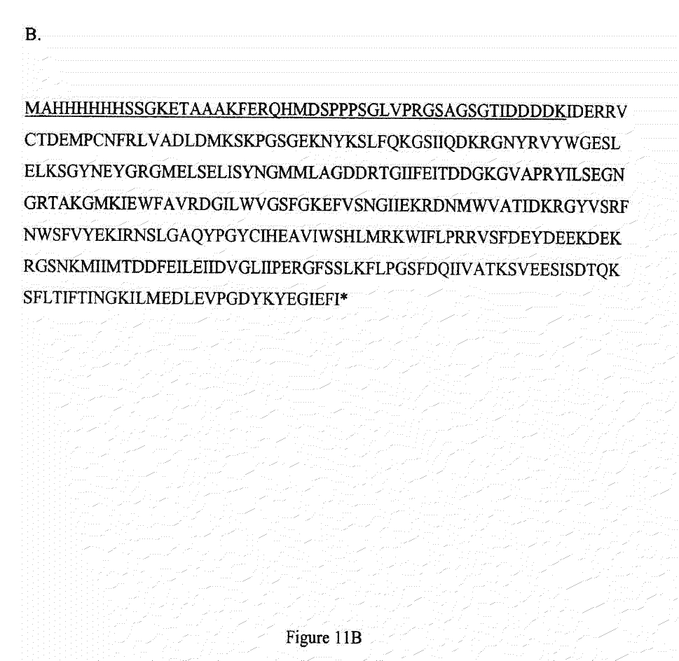

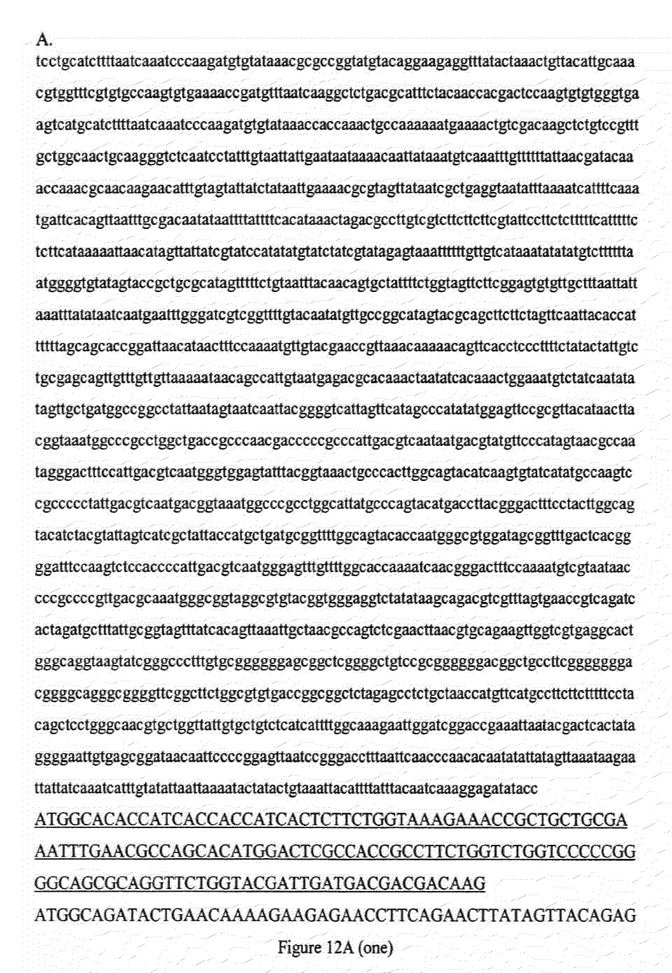

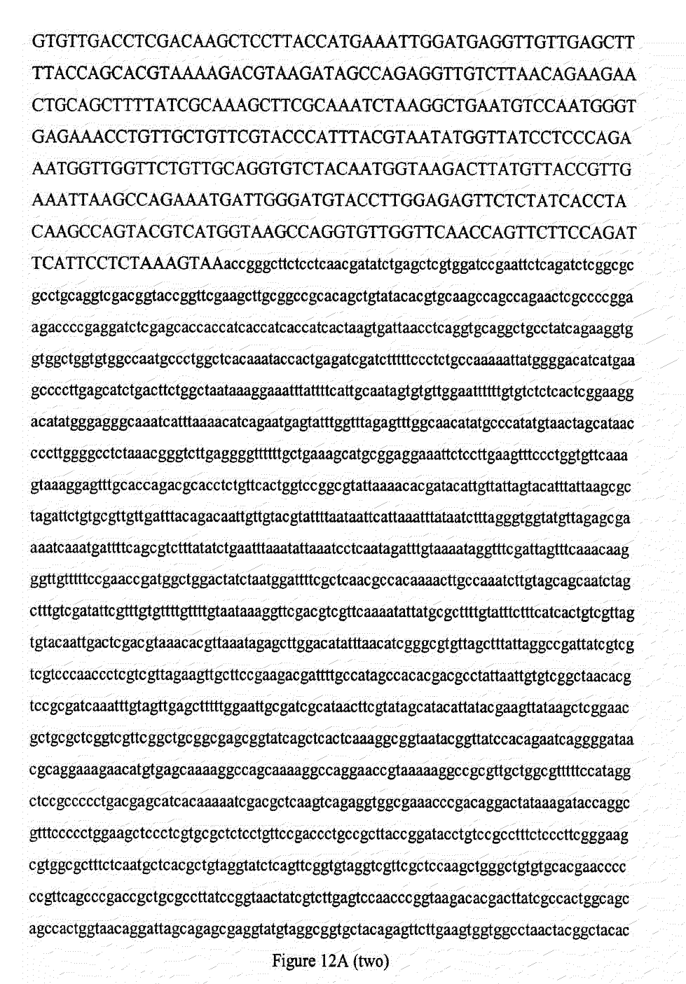

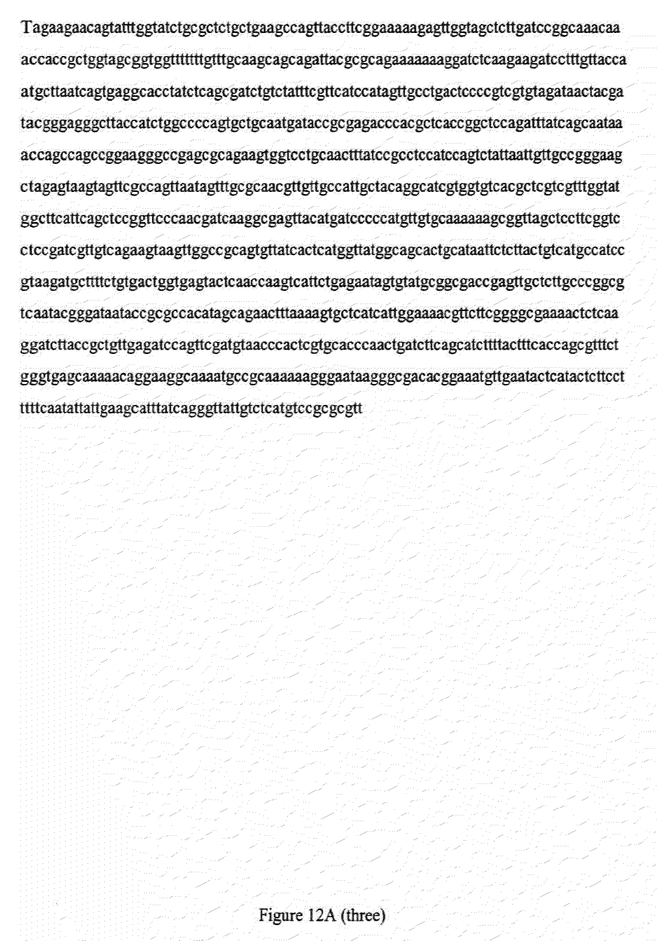

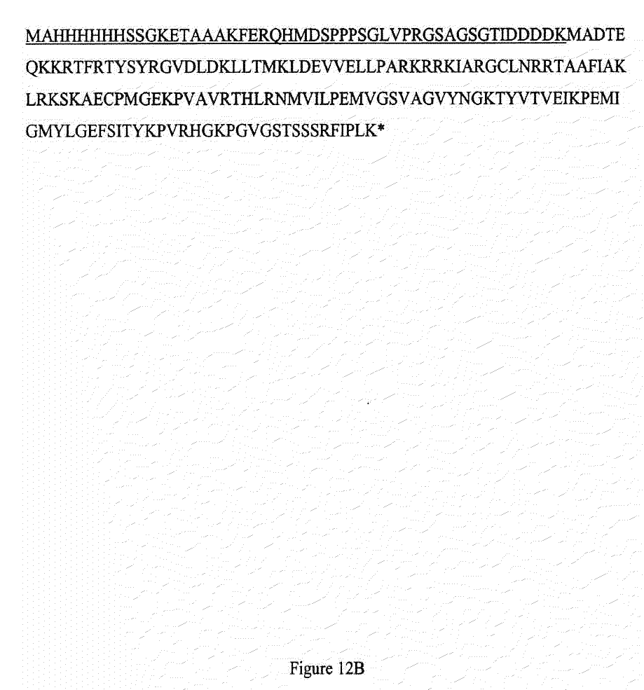

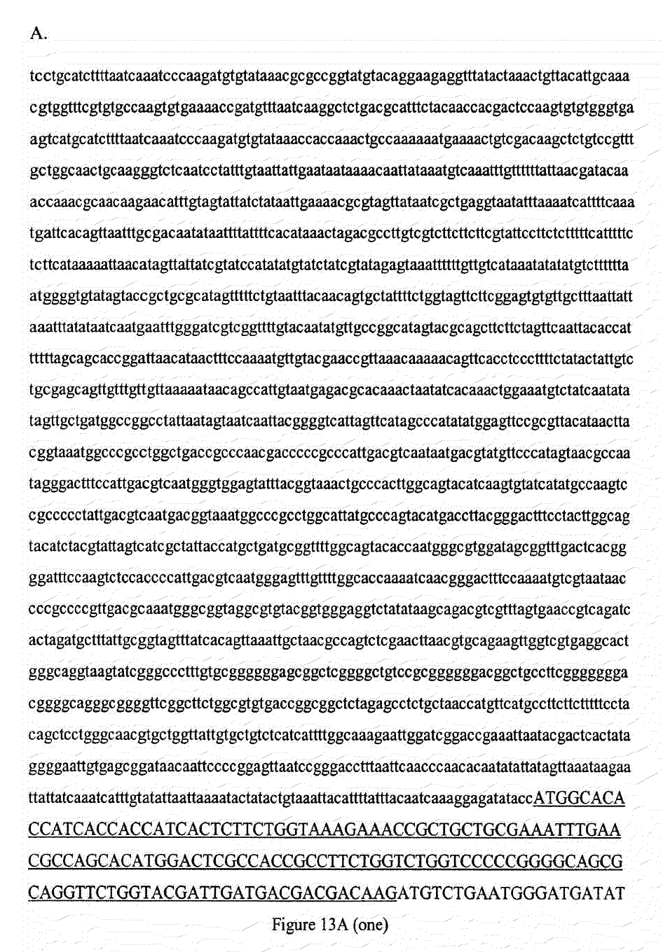

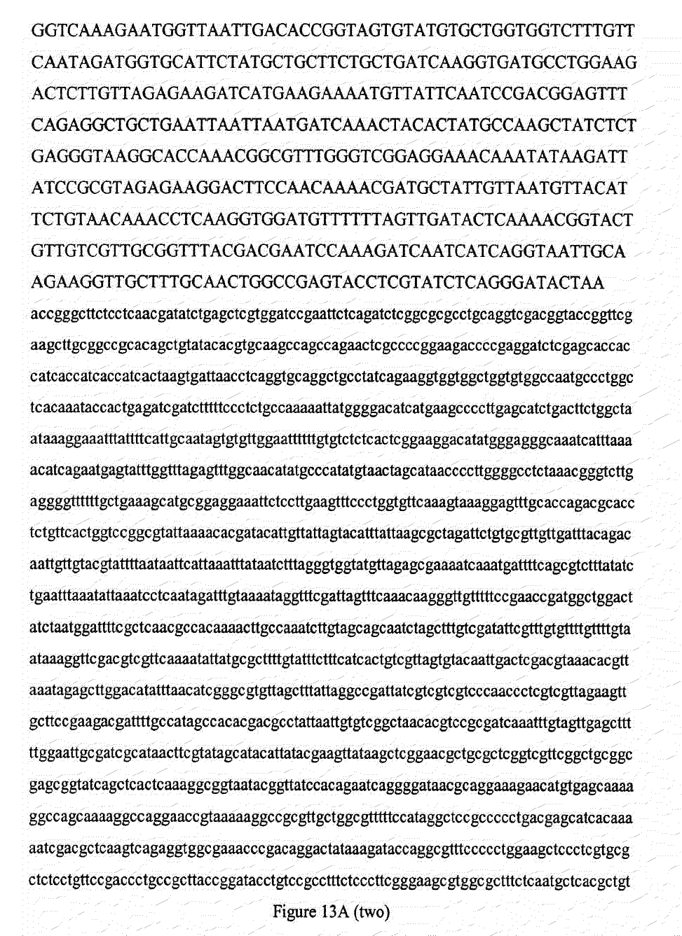

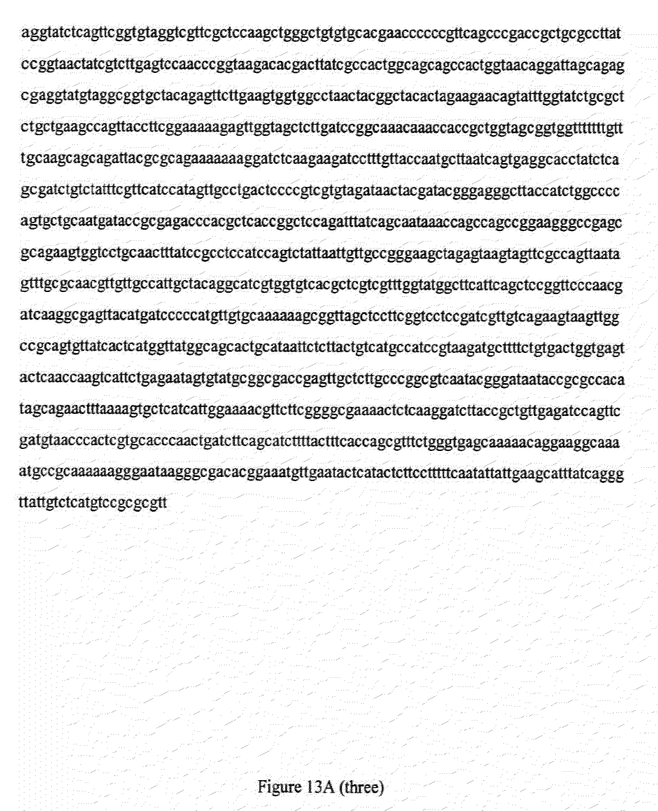

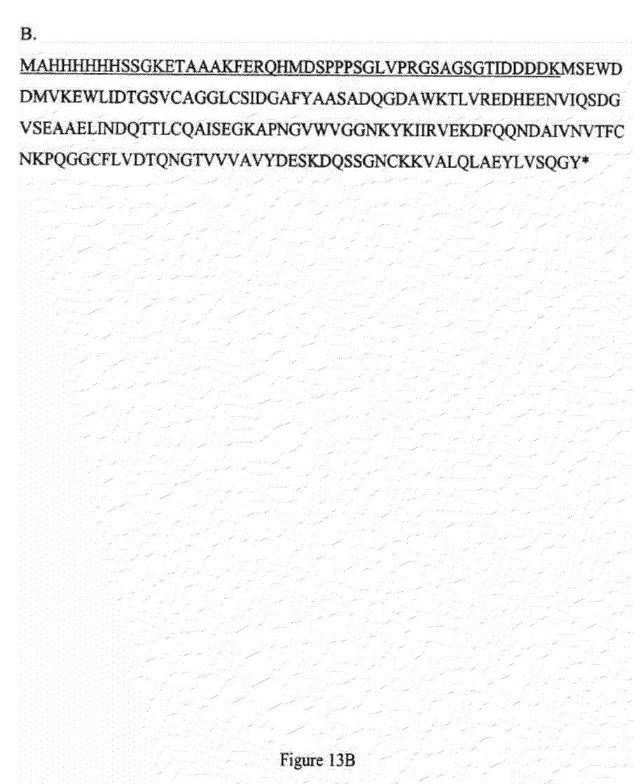

The abilities of these antigens to protect neonatal mice from infections were examined. Each of the above antigens was delivered to 6 day old neonatal mice by intranasal administration of the live Salmonella expressing ClyA fusion proteins. Four days later, the mice were infected with Cryptosporidium oocysts. The infections were followed by quantification of the parasite by qRT-PCR, weight gain, and other characteristics. The results (FIGS. 10A-C) showed that the antigen Cp15 was able to partially protect these animals from the adverse affects of Cryptosporidium infection. This partial protection is remarkable in that a standard immunization protocol usually requires several weeks for optimal induction of immunity whereas these results were observed after only a single dose of ClyA-Cp15 administered in the Salmonella live vector system only 48 hrs before the challenge.

As described above, Cp15 was also tested for its ability to induce an adaptive immune response in normal mice that would protect Rag^{-/-} mice when immune cells or serum was passively transferred. The results (FIG. 9) showed significant reduction in shedding of oocysts after challenge. Thus, this experiment demonstrates that a strong adaptive immune response will protect susceptible individuals from disease and likely reduce shedding of the parasite.

### Example 3

### Experiments Involving the Profilin Antigen

Profilin expressed in bacterial expression systems. The profilin gene was ligated into pTriEX4 (His-Tag), pET41 (GST-Tag), and pET44 (Nus-Tag) E. coli expression vectors and pSEC10 ClyA Salmonella live vaccine vector (as described above) and expressed using standard protocols. The results of these overexpression experiments are summarized in Table 3 and polyacrylamide gel electrophoresis results are presented in FIGS. 4A and B. As can be seen, profilin is expressed very well in pTriEX4 (FIG. 4A, lane 1) and pSEC10 ClyA (FIG. 4B, lane 6).

TABLE 3

| | | pTriEX4 | pET41 | pET44 | pSEC10 |
|---|---|---|---|---|---|
| MW | Func- | (His-Tag) | (GST-Tag) | (Nus-Tag) | ClyA |
| (kDa | tion | cloned | cloned | cloned | Salmonella |
| | | expressed | expressed | expressed | |
| 20 | Cyto- | + Soluble | Not done | + Soluble | + |
| | skeleton | | | | |

These bacterial expression systems thus permit large scale production of profilin protein, and the pSEC10 ClyA fusion vector permits delivery of the antigen in a live secreting bacterial vector intranasally in animal models.

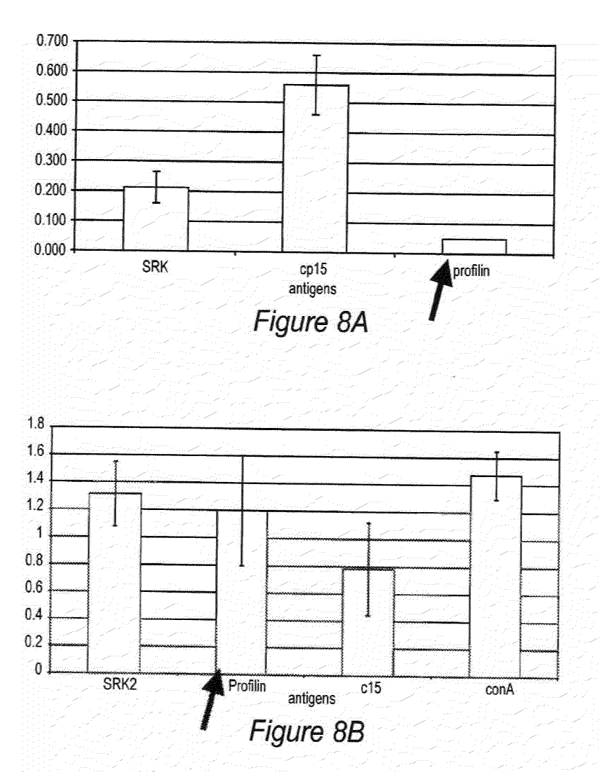

Immunization assays. Interferon gamma knockdown murine model. This model permits infection of adult mice providing much the same symptoms that are seen in human infections. Tests of the profilin protein in this model were performed. Pools of recombinant antigens were used to immunize C57B/6 adult mice in several doses given IM. Anti IFN gamma monoclonal antibody was administered after the final boost, and the mice were challenged with Cryptosporidium. All mice demonstrated a strong humoral immune response against all of the antigens administered, including profilin (FIG. 8, arrow). In contrast, no immune response was detected against any of the candidate antigens in control mice given extracts of Cyrptosporidium (not shown).

Since the Cryptosporidium extracts did induce a humoral response, these results suggest that the selected target antigens, including profilin, are in fact not immunodominant, thus validating a key component of this project.

Antibodies immunolocalized profilin to membrane and block invasion in tissue culture. These antibodies were used to block invasion of cultured human intestinal epithelial cells (HCT8 cell line) by Cryptosporidium and tested for ability to block invasion of cultured HCT-8 cells. The antibodies showed significant inhibitory effects on the rate of infection (not shown). Antibodies were used for immunolocalization and the results confirmed that the proteins are located on or near the cell membrane, and localized to the apical complex (not shown).

Immunization protocols. Immunization protocols using DNA vaccine vectors, recombinant protein, or the intranasal delivery of live vector Salmonella ClyA fusion proteins have been tested, and an efficient protocol involving a combination of intranasal delivery of live vector vaccine and IP delivery of a recombinant protein as a boost was developed. Thus, we tested each of these antigens in adult mice to characterize the IR to each. A protocol in which animals primed (at day 0) with live Salmonella vector (ClyA) expressing Cryptosporidium antigens, at day 14 animals were boosted with either recombinant protein or ClyA expressing Cryptosporidium proteins, and finally at day 28 animals received a second boost with recombinant protein. The results of this immunization protocol showed that all the proteins, including profilin, induced a strong antibody response, with antibody titers increasing dramatically after the first boost. Again, serum from animals immunized with sporozoite extract does not recognize the recombinant protein in dot blot assay, further supporting the above suggestion that these proteins are not immunodominant.

The humoral and cellular immune responses of mice to profilin. Analysis of the immunoglobulin isotypes induced by these immunogens revealed that each antigen induces a unique and distinct pattern, suggesting the possibility of the induction of different T cell populations by these antigens. Significantly, profilin, SRK, and Cp15 induced production of IgA in the intestine as detected by ELISA assay (FIG. 8B, arrow shows profilin). The ability to induce a specific T cell response was also evaluated using a classical lymphoproliferation assay. Profilin, SRK and Cp15 each induced a strong proliferative response with high production of gamma-IFN, a critical cytokine for the control of the disease (FIG. 8B, arrow shows profilin). The abilities of these antigens to protect neonatal mice from infections were examined. Each of the above antigens were delivered to 6 day old neonatal mice by intranasal administration of the live Salmonella expressing ClyA fusion proteins. Four days later, the mice were infected with Cryptosporidium oocysts. The infections were followed by quantification of the parasite by qRT-PCR, weight gain, and other characteristics.

The results showed that profilin was able to partially protect these animals from the adverse affects of Cryptosporidium infection. This partial protection is remarkable in that a standard immunization protocol usually requires several weeks for optimal induction of immunity whereas these results were observed after only a single intranasal inoculation with ClyA-profilin secreting Salmonella live vector approximately 48 hours prior to challenge.

As described above, profilin was also tested for its ability to induce an adaptive immune response in normal mice that would protect Rag−/− mice when immune cells or serum was passively transferred. The results showed significant reduction in shedding of oocysts after challenge. Thus, this experiment demonstrates that a strong adaptive immune response will protect susceptible individuals from disease and likely reduce shedding of the parasite.

### REFERENCES

1. Goodgame R W. Understanding intestinal spore-forming protozoa: cryptosporidia, microsporidia, isospora, and cyclospora. Ann. Intern. Med. 1996;124(4):429-41.
2. EPA office of water. Cryptosporidium Spp. systematics and waterborne challenges in public health. Summary Report 1999. 2002.
3. Korich D G, Mead J R, Madore M S, Sinclair N A, Sterling C R. Effects if ozone, chlorine dioxide, chlorine, and monochloramine on Cryptosporidium parvum oocyst viability. Appl Environ Microbial. 1990; 56: 1423-1428.
4. MacKenzie W R, Hoxie, N J, Proctor M E et al. A massive outbreak in Milwaukee of Cryptosporidium infection transmitted through the public water supply. N Engl J. Med. 1994; 331: 161-167.
5. Pozie E, Rezza G. Boschini A et al. Clinical cryptosporidiosis and human immunodeficiency virus-induced immunosuppression: findings from a longitudinal study of HIV-positive and HIV-negative former injection drug users. J Infect Dis. 1997; 176:969-975.
6. Jenkins M, and Fayer, R. Cloning and expression of cDNA encoding an antigenic Cryptosporidium parvum protein. Mol. Bioch. Parasitol. 1995; 149-152
7. Riggs M. W. Recent advances in cryptosporidiosis: the immune response. Microb. Infect. 2002; 1067-1080
8. Singh I., Theodos C. and Tzipori S. Recombinant Proteins of Cryptosporidium parvum induce proliferation of mesenteric lymph node cells in infected mice. Infec. Imm. 2005; 73: 5245-5248.
9. Xu, P., Widmer, G., Wang, Y., Ozaki, L. S., Alves, J., Serrano, M., Puiu, D., Manque, P., Akiyoshi, D., Mackey, A., Pearson, W., Dear, P., Bankier, A., Peterson, D., Abrahamsen, M S., Kapur, V., Tzipori, S., and Buck, G A. The Genome of Cryptosporidium hominis. Nature 431: 1107-1112 (2004).
10. Yarovinsky F, Zhang D, Andersen J F, Bannenberg G L, Serhan C N, Hayden M S, Hieny S, Sutterwala F S, Flavell R A, Ghosh S, Sher A. TLR11 activation of dendritic cells by a protozoan profilin-like protein. Science 2005; 308(5728) 1626-29.
11. Abrahamsen, M. S. (2004). “Complete Genome Sequence of the Apicomplexan, Cryptosporidium parvum”. Science 304: 441

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A composition comprising
one or more recombinant amino acid sequences as set forth in SEQ ID NO: 1, SEQ ID NO: 3, and SEQ ID NO: 5, or variants thereof; or
one or more nucleic acid sequences encoding said one or more recombinant amino acid sequences of said variants; and
a physiological compatible carrier.

2. The composition of claim 1, wherein said nucleic acid sequences are present within a vector.

3. The composition of claim 2, wherein said vector is a Salmonella based vector pSEC 10 ClyA.

4. A method of vaccinating a subject against Cryptosporidiosis, comprising the step of
1) providing to said subject a composition comprising one or more recombinant proteins with amino acid sequences set forth in SEQ ID NO: 1, SEQ ID NO: 3, and SEQ ID NO: 5, or variants thereof, and a physiologically compatible carrier; or
2) providing to said subject a composition comprising one or more nucleic acid sequences encoding said one or more recombinant proteins with amino acid sequences set forth in SEQ ID NO: 1, SEQ ID NO: 3, and SEQ ID NO: 5, or variants thereof, and a physiologically compatible carrier; or
3) sequentially providing to said subject
i. a composition comprising one or more nucleic acid sequences encoding said one or more recombinant proteins with amino acid sequences set forth in SEQ ID NO: 1, SEQ ID NO: 3, and SEQ ID NO: 5, or variants thereof; and a physiologically compatible carrier; and
ii. a composition comprising one or more recombinant proteins with amino acid sequences set forth in SEQ ID NO: 1, SEQ ID NO: 3, and SEQ ID NO: 5, or variants thereof; and a physiologically compatible carrier;
wherein said composition is or said compositions are provided in a quantity sufficient to protect said subject against infection by Cryptosporidium, or to lessen symptoms of Cryptosporidiosis is said subject.

5. The method of claim 4, wherein said nucleic acids are present within a vector.

6. The method of claim 5, wherein said vector is a Salmonella based vector.

7. The method of claim 6, wherein said Salmonella based vector is administered intranasally.

8. A method of decreasing the shedding of Cryptosporidium oocysts by a subject infected with Cryptosporidium, comprising the step of
1) providing to said subject a composition comprising one or more recombinant proteins with amino acid sequences set forth in SEQ ID NO: 1, SEQ ID NO: 3, and SEQ ID NO: 5, or variants thereof, and a physiologically compatible carrier; or
2) providing to said subject a composition comprising one or more nucleic acid sequences encoding said one or more recombinant proteins with amino acid sequences set forth in SEQ ID NO: 1, SEQ ID NO: 3, and SEQ ID NO: 5, or variants thereof, and a physiologically compatible carrier; or
3) sequentially providing to said subject
i. a composition comprising one or more nucleic acid sequences encoding said one or more recombinant proteins with amino acid sequences set forth in SEQ ID NO: 1, SEQ ID NO: 3, and SEQ ID NO: 5, or variants thereof, and a physiologically compatible carrier; and
ii. a composition comprising one or more recombinant proteins with amino acid sequences set forth in SEQ ID NO: 1, SEQ ID NO: 3, and SEQ ID NO: 5, or variants thereof, and a physiologically compatible carrier;
wherein said composition is or said compositions are provided in a quantity sufficient to reduce the number of Cryptosporidium oocysts shed by said subject.

9. The method of claim 8, wherein said nucleic acids are present within a vector.

10. The composition of claim 8, wherein said vector is a Salmonella based vector.

11. The method of claim 10, wherein said Salmonella based vector is provided intranasally.

12. A composition for use as an adjuvant, comprising
a protein with an amino acid sequence as set forth in SEQ ID NO: 5, or a nucleic acid sequence encoding said protein with an amino acid sequence as set forth in SEQ ID NO: 5, or a variant thereof; and
a physiologically compatible carrier.

13. A method of increasing an immune response to an antigen in a subject in need thereof, comprising the step of providing to said subject
i) said antigen of interest or a nucleic acid encoding said antigen of interest; and
ii) a protein with an amino acid sequence as set forth in SEQ ID NO: 5 or a variant thereof, or
a nucleic acid sequence encoding said protein with an amino acid sequence as set forth in SEQ ID NO: 5 or a variant thereof;
wherein said protein with an amino acid sequence as set forth in SEQ ID NO: 5 or a variant thereof thereof or said nucleic acid sequence encoding said protein with an amino acid sequence as set forth in SEQ ID NO: 5 or or a variant thereof thereof is provided in a quantity sufficient to cause an increase in said immune response to said antigen of interest in said subject.

14. A method of vaccinating a subject against Cryptosporidiosis, comprising the step of
providing aid subject with one or more sporozoite antigens or one or more vectors containing nucleotides coding for said one or more sporozoite antigens.

15. The method of claim 14, wherein said one or more sporozoite antigens is selected from the group consisting of SRK, CP15 and profilin from Cryptosporidium.


SEQUENCE LISTING (12 sequences)

SEQ ID NO: 1 - protein, 345 aa, Cryptosporidium hominis
MKESGTINYLITFTFIIPFVLSQSTLLNLGAGGIQERRVCTDEMPCNFRLVADLDMKSKPGSGEKNYKSLFQKGSIIQDKRGNYRVYWGESLELKSGYNEYGRGMELSELISYNGMMLAGDDRTGIIFEITDDGKGVAPRYILSEGNGRTAKGMKIEWFAVRDGILWVGSFGKEFVSNGIIEKRDNMWVATIDKRGYVSRFNWSFVYEKIRNSLGAQYPGYCIHEAVIWSHLMRKWIFLPRRVSFDEYDEEKDEKRGSNKMIIMTDDFEILEIIDVGLIIPERGFSSLKFLPGSFDQIIVATKSVEESISDTQKSFLTIFTINGKILMEDLEVPGDYKYEGIEFI

SEQ ID NO: 2 - DNA, 1038 bp, Cryptosporidium hominis
atgaaagaatcaggcacaattaattatctaataacatttacattcattattcctttcgtactttcccagtcaacattattaaatcttggtgcagggggtatacaggaaaggagggtttgcactgacgaaatgccatgtaactttagattggttgctgatttagatatgaagtcaaagccagggagtggggaaaagaattacaaaagtttatttcaaaaagggtcaataatacaagacaaaaggggcaactatcgagtgtactggggagaaagtctggaacttaaaagcggatataatgaatatgggagagggatggaattaagtgagttgatttcatataatggaatgatgcttgcgggcgacgaccgtacaggaataatttttgaaataactgatgatggaaaaggagtagcaccaagatatatattatctgaaggtaatggaagaacagctaagggaatgaagattgagtggtttgctgtaagagatggaatattgtgggttggcagttttggaaaagagttcgtatcaaacggcataatagaaaaaagagataatatgtgggtagccacaattgataaaagaggatatgtttcacgatttaattggagttttgtttatgaaaaaattaggaattcactgggggcgcaatatccaggttattgcattcatgaagcagtgatttggagtcatttaatgagaaagtggatatttttaccaagaagagttagcttcgatgagtatgatgaggagaaagacgaaaagagaggttccaataaaatgataattatgacagatgattttgaaattcttgaaattattgacgtaggattgataatacctgaaagaggtttttcttctttaaaatttcttcctgggtcgtttgaccagataatagttgcaacaaaaagcgttgaagaatcaatttcagacactcaaaagtctttcttaactatattcacaataaatggaaaaattttaatggaagatttagaagtgcctggagactacaaatacgaggggatagaatttatatag

SEQ ID NO: 3 - protein, 144 aa, Cryptosporidium hominis
ADTEQKKRTFRTYSYRGVDLDKLLTMKLDEVVELLPARKRRKIARGCLNRRTAAFIAKLRKSKAECPMGEKPVAVRTHLRNMVILPEMVGSVAGVYNGKTYVTVEIKPEMIGMYLGEFSITYKPVRHGKPGVGSTSSSRFIPLK

SEQ ID NO: 4 - DNA, 438 bp, cryptosporidium hominis
atggcagatactgaacaaaagaagagaaccttcagaacttatagttacagaggtgttgacctcgacaagctccttaccatgaaattggatgaggttgttgagcttttaccagcacgtaaaagacgtaagatagccagaggttgtcttaacagaagaactgcagcttttatcgcaaagcttcgcaaatctaaggctgaatgtccaatgggtgagaaacctgttgctgttcgtacccatttacgtaatatggttatcctcccagaaatggttggttctgttgcaggtgtctacaatggtaagacttatgttaccgttgaaattaagccagaaatgattgggatgtaccttggagagttctctatcacctacaagccagtacgtcatggtaagccaggtgttggttcaaccagttcttccagattcattcctctaaagtaa

SEQ ID NO: 5 - protein, 162 aa, Cryptosporidium hominis
MSEWDDMVKEWLIDTGSVCAGGLCSIDGAFYAASADQGDAWKTLVREDHEENVIQSDGVSEAAELINDQTTLCQAISEGKAPNGVWVGGNKYKIIRVEKDFQQNDAIVNVTFCNKPQGGCFLVDTQNGTVVVAVYDESKDQSSGNCKKVALQLAEYLVSQGY

SEQ ID NO: 6 - DNA, 489 bp, Cryptosporidium hominis
atgtctgaatgggatgatatggtcaaagaatggttaattgacaccggtagtgtatgtgctggtggtctttgttcaatagatggtgcattctatgctgcttctgctgatcaaggtgatgcctggaagactcttgttagagaagatcatgaagaaaatgttattcaatccgacggagtttcagaggctgctgaattaattaatgatcaaactacactatgccaagctatctctgagggtaaggcaccaaacggcgtttgggtcggaggaaacaaatataagattatccgcgtagagaaggacttccaacaaaacgatgctattgttaatgttacattctgtaacaaacctcaaggtggatgttttttagttgatactcaaaacggtactgttgtcgttgcggtttacgacgaatccaaagatcaatcatcaggtaattgcaagaaggttgctttgcaactggccgagtacctcgtatctcagggatactaa

SEQ ID NO: 7 - DNA, 6175 bp, Artificial
  Plasmid pTriEx-4/60194 containing sequences encoding SRK
tcctgcatcttttaatcaaatcccaagatgtgtataaacgcgccggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgtcaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcttcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatggccggcctattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatgctgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagacgtcgtttagtgaaccgtcagatcactagatgctttattgcggtagtttatcacagttaaattgctaacgccagtctcgaacttaacgtgcagaagttggtcgtgaggcactgggcaggtaagtatcgggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcggaccgaaattaatacgactcactataggggaattgtgagcggataacaattccccggagttaatccgggacctttaattcaacccaacacaatatattatagttaaataagaattattatcaaatcatttgtatattaattaaaatactatactgtaaattacattttatttacaatcaaaggagatataccatggcacaccatcaccaccatcactcttctggtaaagaaaccgctgctgcgaaatttgaacgccagcacatggactcgccaccgccttctggtctggtcccccggggcagcgcaggttctggtacgattgatgacgacgacaagatagacgaaaggagggtttgcactgacgaaatgccatgtaactttagattggttgctgatttagatatgaagtcaaagccagggagtggggaaaagaattacaaaagtttatttcaaaaagggtcaataatacaagacaaaaggggcaactatcgagtgtactggggagaaagtctggaacttaaaagcggatataatgaatatgggagagggatggaattaagtgagttgatttcatataatggaatgatgcttgcgggcgacgaccgtacaggaataatttttgaaataactgatgatggaaaaggagtagcaccaagatatatattatctgaaggtaatggaagaacagctaagggaatgaagattgagtggtttgctgtaagagatggaatattgtgggttggcagttttggaaaagagttcgtatcaaacggcataatagaaaaaagagataatatgtgggtagccacaattgataaaagaggatatgtttcacgatttaattggagttttgtttatgaaaaaattaggaattcactgggggcgcaatatccaggttattgcattcatgaagcagtgatttggagtcatttaatgagaaagtggatatttttaccaagaagagttagcttcgatgagtatgatgaggagaaagacgaaaagagaggttccaataaaatgataattatgacagatgattttgaaattcttgaaattattgacgtaggattgataatacctgaaagaggtttttcttctttaaaatttcttcctgggtcgtttgaccagataatagttgcaacaaaaagcgttgaagaatcaatttcagacactcaaaagtctttcttaactatattcacaataaatggaaaaattttaatggaagatttagaagtgcctggagactacaaatacgaggggatagaatttatataaaccgggcttctcctcaacgatatctgagctcgtggatccgaattctcagatctcggcgcgcctgcaggtcgacggtaccggttcgaagcttgcggccgcacagctgtatacacgtgcaagccagccagaactcgccccggaagaccccgaggatctcgagcaccaccatcaccatcaccatcactaagtgattaacctcaggtgcaggctgcctatcagaaggtggtggctggtgtggccaatgccctggctcacaaataccactgagatcgatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgtaactagcataaccccttggggcctctaaacgggtcttgaggggttttttgctgaaagcatgcggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaatagagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattgcgatcgcataacttcgtatagcatacattatacgaagttataagctcggaacgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgtccgcgcgtt

SEQ ID NO: 8 - protein, 360 aa, Cryptosporidium hominis
MAHHHHHHSSGKETAAAKFERQHMDSPPPSGLVPRGSAGSGTIDDDDKIDERRVCTDEMPCNFRLVADLDMKSKPGSGEKNYKSLFQKGSIIQDKRGNYRVYWGESLELKSGYNEYGRGMELSELISYNGMMLAGDDRTGIIFEITDDGKGVAPRYILSEGNGRTAKGMKIEWFAVRDGILWVGSFGKEFVSNGIIEKRDNMWVATIDKRGYVSRFNWSFVYEKIRNSLGAQYPGYCIHEAVIWSHLMRKWIFLPRRVSFDEYDEEKDEKRGSNKMIIMTDDFEILEIIDVGLIIPERGFSSLKFLPGSFDQIIVATKSVEESISDTQKSFLTIFTINGKILMEDLEVPGDYKYEGIEFI

SEQ ID NO: 9 - DNA, 5674 bp, Artificial
  Plasmid pTriEx-4/60368 containing sequences encoding Cp15
tcctgcatcttttaatcaaatcccaagatgtgtataaacgcgccggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgtcaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcttcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatggccggcctattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatgctgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagacgtcgtttagtgaaccgtcagatcactagatgctttattgcggtagtttatcacagttaaattgctaacgccagtctcgaacttaacgtgcagaagttggtcgtgaggcactgggcaggtaagtatcgggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcggaccgaaattaatacgactcactataggggaattgtgagcggataacaattccccggagttaatccgggacctttaattcaacccaacacaatatattatagttaaataagaattattatcaaatcatttgtatattaattaaaatactatactgtaaattacattttatttacaatcaaaggagatataccatggcacaccatcaccaccatcactcttctggtaaagaaaccgctgctgcgaaatttgaacgccagcacatggactcgccaccgccttctggtctggtcccccggggcagcgcaggttctggtacgattgatgacgacgacaagatggcagatactgaacaaaagaagagaaccttcagaacttatagttacagaggtgttgacctcgacaagctccttaccatgaaattggatgaggttgttgagcttttaccagcacgtaaaagacgtaagatagccagaggttgtcttaacagaagaactgcagcttttatcgcaaagcttcgcaaatctaaggctgaatgtccaatgggtgagaaacctgttgctgttcgtacccatttacgtaatatggttatcctcccagaaatggttggttctgttgcaggtgtctacaatggtaagacttatgttaccgttgaaattaagccagaaatgattgggatgtaccttggagagttctctatcacctacaagccagtacgtcatggtaagccaggtgttggttcaaccagttcttccagattcattcctctaaagtaaaccgggcttctcctcaacgatatctgagctcgtggatccgaattctcagatctcggcgcgcctgcaggtcgacggtaccggttcgaagcttgcggccgcacagctgtatacacgtgcaagccagccagaactcgccccggaagaccccgaggatctcgagcaccaccatcaccatcaccatcactaagtgattaacctcaggtgcaggctgcctatcagaaggtggtggctggtgtggccaatgccctggctcacaaataccactgagatcgatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgtaactagcataaccccttggggcctctaaacgggtcttgaggggttttttgctgaaagcatgcggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaatagagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattgcgatcgcataacttcgtatagcatacattatacgaagttataagctcggaacgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgtccgcgcgtt

SEQ ID NO: 10 - protein, 193 aa, Cryptosporidium hominis
MAHHHHHHSSGKETAAAKFERQHMDSPPPSGLVPRGSAGSGTIDDDDKMADTEQKKRTFRTYSYRGVDLDKLLTMKLDEVVELLPARKRRKIARGCLNRRTAAFIAKLRKSKAECPMGEKPVAVRTHLRNMVILPEMVGSVAGVYNGKTYVTVEIKPEMIGMYLGEFSITYKPVRHGKPGVGSTSSSRFIPLK

SEQ ID NO: 11 - DNA, 5725 bp, Artificial
  Plasmid pTriEx-4/30189 containing sequences encoding Profilin
tcctgcatcttttaatcaaatcccaagatgtgtataaacgcgccggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgtcaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcttcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatggccggcctattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatgctgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagacgtcgtttagtgaaccgtcagatcactagatgctttattgcggtagtttatcacagttaaattgctaacgccagtctcgaacttaacgtgcagaagttggtcgtgaggcactgggcaggtaagtatcgggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcggaccgaaattaatacgactcactataggggaattgtgagcggataacaattccccggagttaatccgggacctttaattcaacccaacacaatatattatagttaaataagaattattatcaaatcatttgtatattaattaaaatactatactgtaaattacattttatttacaatcaaaggagatataccatggcacaccatcaccaccatcactcttctggtaaagaaaccgctgctgcgaaatttgaacgccagcacatggactcgccaccgccttctggtctggtcccccggggcagcgcaggttctggtacgattgatgacgacgacaagatgtctgaatgggatgatatggtcaaagaatggttaattgacaccggtagtgtatgtgctggtggtctttgttcaatagatggtgcattctatgctgcttctgctgatcaaggtgatgcctggaagactcttgttagagaagatcatgaagaaaatgttattcaatccgacggagtttcagaggctgctgaattaattaatgatcaaactacactatgccaagctatctctgagggtaaggcaccaaacggcgtttgggtcggaggaaacaaatataagattatccgcgtagagaaggacttccaacaaaacgatgctattgttaatgttacattctgtaacaaacctcaaggtggatgttttttagttgatactcaaaacggtactgttgtcgttgcggtttacgacgaatccaaagatcaatcatcaggtaattgcaagaaggttgctttgcaactggccgagtacctcgtatctcagggatactaaaccgggcttctcctcaacgatatctgagctcgtggatccgaattctcagatctcggcgcgcctgcaggtcgacggtaccggttcgaagcttgcggccgcacagctgtatacacgtgcaagccagccagaactcgccccggaagaccccgaggatctcgagcaccaccatcaccatcaccatcactaagtgattaacctcaggtgcaggctgcctatcagaaggtggtggctggtgtggccaatgccctggctcacaaataccactgagatcgatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgtaactagcataaccccttggggcctctaaacgggtcttgaggggttttttgctgaaagcatgcggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaatagagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattgcgatcgcataacttcgtatagcatacattatacgaagttataagctcggaacgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgtccgcgcgtt

SEQ ID NO: 12 - protein, 210 aa, Cryptosporidium hominis
MAHHHHHHSSGKETAAAKFERQHMDSPPPSGLVPRGSAGSGTIDDDDKMSEWDDMVKEWLIDTGSVCAGGLCSIDGAFYAASADQGDAWKTLVREDHEENVIQSDGVSEAAELINDQTTLCQAISEGKAPNGVWVGGNKYKIIRVEKDFQQNDAIVNVTFCNKPQGGCFLVDTQNGTVVVAVYDESKDQSSGNCKKVALQLAEYLVSQGY